(19) United States
(12) Patent Application Publication
(10) Pub. No.: US 2024/0342417 A1
(43) Pub. Date: Oct. 17, 2024

(54) CUSHION FOR A RESPIRATORY MASK AND RESPIRATORY MASK HAVING THE SAME

(71) Applicant: Fisher & Paykel Healthcare Limited, Auckland (NZ)

(72) Inventors: Mark Andrew THOMPSON, Auckland (NZ); Bruno SINTIVE, Queensland (AU)

(21) Appl. No.: 18/569,186

(22) PCT Filed: Jul. 6, 2022
(86) PCT No.: PCT/IB2022/056225

§ 371 Date: Dec. 11, 2023

Related U.S. Application Data
(60) Provisional application No. 63/203,057, filed on Jul. 6, 2021.
(60) Provisional application No. 63/366,973, filed on Jun. 24, 2022.

(51) Int. Cl. A61M 16/06

(52) U.S. Cl. CPC A61M 16/0622; A61M 2210/0618

(57) ABSTRACT

A nasal cushion for a respiratory interface has a central portion and left and right wings that extend to respective left and right lateral ends from the central portion. The cushion has an inner cushion wall to contact a patient's face and a non-face contacting outer cushion wall. A connecting wall connects the inner cushion wall and outer cushion wall. The cushion also has a bellows extending in length along the connecting wall and in depth into the cushion at each of the left and right wings of the cushion. Each of the bellows are expandable to increase a spacing between the inner cushion wall and outer cushion wall.

### FIELD OF TECHNOLOGY

The present disclosure generally relates to a respiratory mask system for the delivery of respiratory therapy to a patient.

More particularly but not solely, the present disclosure relates to a cushion for a respiratory mask.

### BACKGROUND

Respiratory masks are used to provide respiratory therapy through the delivery under positive pressure of a gas or gases to the airways of a person suffering from various of respiratory illnesses or conditions. Such therapies may include but are not limited to continuous positive airway pressure (CPAP) therapy and non-invasive ventilation (NIV) therapy.

CPAP therapy can be used to treat obstructive sleep apnea (OSA), which is a condition in which a patient's airway intermittently collapses, during sleep, preventing the patient from breathing for a period of time. The cessation of breathing, or apnea, results in the patient awakening. Repetitive and frequent apneas may result in the patient rarely achieving a full and restorative night's sleep.

CPAP therapy involves the delivery of a supply of continuous positive air pressure to the airway of the patient via a respiratory mask. The continuous positive pressure acts as a splint within the patient's airway, which secures the airway in an open position such that the patient's breathing and sleep are not interrupted.

Respiratory masks typically comprise a patient interface and a headgear, wherein the patient interface is configured to deliver the supply of continuous positive air pressure to the patient's airway via a seal or cushion that forms a substantially airtight seal in or around the patient's nose and/or mouth.

Respiratory masks are available in a range of styles including full-face, nasal, direct nasal and oral masks, which create a substantially airtight seal with the nose and/or mouth. The seal or cushion is held in place on the patient's face by headgear. In order to maintain a substantially airtight seal the headgear should provide support to the patient interface such that it is held in a stable position relative to the patient's face during use. Such respiratory masks may also be used to deliver NIV, CPAP, and other therapies.

### SUMMARY

According to a first aspect, the present disclosure provides for a nasal cushion for a respiratory interface, the cushion having a central portion and left and right wings that extend to respective left and right lateral ends from the central portion, the cushion comprising:
an inner cushion wall to contact a patient's face,
a non-face contacting outer cushion wall,
a connecting wall that connects the inner cushion wall and outer cushion wall, and
a bellows extending in length along the connecting wall and in depth into the cushion at each of the left and right wings of the cushion,
wherein the or each of the bellows are expandable to increase a spacing between the inner cushion wall and outer cushion wall.

The bellows extending along the connecting wall at each of the left and right wings of the cushion is a single unitary bellows.

The bellows extending along the connecting wall at each of the left and right wings of the cushion comprise a first bellows of the left wing and a second bellows of a right wing of the cushion.

The first bellows and second bellows are each provided along the upper portion of the connecting wall but are spaced apart from each other at the central portion.

The first bellows and second bellows are spaced apart across the central portion by about 1 mm to about 30 mm.

The first bellows and second bellows are spaced apart across the central portion by about 1 mm to about 16 mm.

The or each of the bellows respectively extend in length from each of the left and right lateral ends of the cushion along the left and right wings towards the central portion.

The or each of the bellows respectively extend in length in an at least partially distal direction from each of the left and right lateral ends of the cushion.

The or each of the bellows respectively extend in length both distally and medially from each of the left and right lateral ends of the cushion.

The or each of the bellows respectively extend in depth into the cushion in a distal direction from each of the left and right lateral ends of the cushion.

The connecting wall includes an upper portion connecting respective upper parts of the inner cushion wall and the outer cushion wall and a lower portion connecting respective lower parts of the inner cushion wall and the outer cushion wall, and the or each of the bellows extend in length along the upper portion of the connecting wall.

The or each of the bellows extend in length along both the upper portion and lower portion of the connecting wall.

The or each of the bellows extend in length along the connecting wall around respective left and right lateral ends of the cushion between the or a upper portion and the or a lower portion of the connecting wall.

The or each of the bellows extend in length along the upper portion of the connecting wall and the respective left and right ends of the cushion such that a proximal end of the or each of the bellows is located closer to the lower portion of the connecting wall than to the upper portion of the connecting wall.

Each of the left and right lateral ends of the cushion have a proximal tip relative to a patient in use, and the or each of the bellows extend in length along the upper portion of the connecting wall above each of the respective proximal tips and terminate below each of the respective proximal tips.

The or each of the bellows extend in length along the lower portion of the connecting wall more distally than an alar facial groove of a patient in use.

The or each of the bellows comprise an inner bellows wall, an outer bellows wall, and a bellows walls joint connecting the inner and outer bellows walls.

The or each inner bellows wall is adjacent to the inner cushion wall and the or each outer bellows wall is adjacent to the outer cushion wall.

The or each of the inner bellows walls and outer bellows walls extend at one end from the connecting wall and at the other end from a respective bellows walls joint.

the or each of the inner bellows walls and outer bellows walls extend at one end from the upper portion of the connecting wall and at the other end from a respective bellows walls joint.

In an at rest condition, the or each of the internal and external bellows walls extend into the cushion in a space between the internal cushion wall and external cushion wall.

The or each bellows walls joint comprises a curved joint wall extending between respective inner and outer bellows walls.

The or each bellows walls joint extends tangentially from each of an associated inner and outer bellows walls and is continuously curved therebetween.

A surface of at least one portion of the or each bellows walls joint at an upper portion of the cushion has a first radius of curvature, and a surface of at least one portion of the or each bellows walls joint at a lower portion of the cushion has a second radius of curvature.

The second radius of curvature is greater than the first radius of curvature.

The second radius of curvature is about 5% to about 25% greater than the first radius of curvature.

The second radius of curvature is about 1.6 mm, and the first radius of curvature is about 1.4 mm.

In an at rest condition there is a substantially uniform spacing between the inner and outer bellows walls of the or each of the bellows.

In an at rest condition, the inner and outer bellows walls of the or each of the bellows are substantially plane-parallel with each other.

In an at rest condition, the inner and outer bellows walls of each bellows diverge from each other away from a base of the bellows.

A spacing between the inner and outer bellows walls of the or each of the bellows is about 0.5 mm to about 2 mm.

In an at rest condition, the inner and outer bellows walls of the or each of the bellows are at least partially in contact with each other.

The or each bellows walls joint has a relatively greater thickness than each of the associated inner and outer bellows walls.

The or each bellows walls joint has a relatively greater stiffness than each of the associated inner and outer bellows walls.

An upper bellows joint is defined between the or each of the inner and outer bellows walls and the connecting wall, and each upper bellows joint has a relatively greater thickness than each of the associated inner and outer bellows walls.

Each of the upper bellows joint of the or each of the bellows has a relatively greater stiffness than each of the inner and outer bellows walls of the respective bellows.

Each of the inner and outer bellows walls are relatively thicker than at least an adjacent part of the inner cushion wall.

A thickness of each of the inner and outer bellows walls is substantially uniform.

A or the thickness of each the inner and outer bellows walls of each bellows are substantially equal.

A or the thickness of each of the inner and outer bellows walls is about 0.3 mm to about 1 mm.

A or the thickness of each of the inner and outer bellows walls is about 0.5 mm.

The thickness of each of the inner and outer bellows walls is greater at a lower portion of the connecting wall than at an upper portion of the connecting wall.

The or each of the bellows comprise a first region in a central portion of the cushion and a second region extending to a proximal opening of the bellows, wherein an orientation of the bellows along their length changes from the first region of the bellows to the second region of the bellows.

In the first region the or each of the bellows extend in length proximally and curve medially along the connecting wall from the central portion of the cushion.

In the second region of the or each of the bellows, towards the proximal opening of the bellows, the bellows extend laterally.

The proximal opening of the or each of the bellows is located towards a lateral side of each respective wing of the cushion.

The proximal opening of the or each of the bellows is located closer to the outer cushion wall than to the inner cushion wall.

The length of the or each of the bellows is between a first end and a second end of the or each of the bellows along the connecting wall and the depth of the or each of the bellows is between an associated bellows walls joint and the connecting wall, and the depth of the or each of the bellows varies between its first end and its second end.

The depth of the or each of the bellows increases away from its first end and second end.

The depth of the or each of the bellows reduces to zero at one or both of its respective first end and second end.

The depth of the or each of the bellows between its first and second ends is greatest at a region to be located between a supra-alar crease and an alar base of a patient's nose.

The depth of at least one of the bellows between its first and second ends is greatest at a region to be located adjacent the alar groove of the a patient's nose.

The or each of the bellows extend in length around the left and right ends of the cushion, and a radius of curvature of the connecting wall at each of the left and right ends of the cushion is greater than a radius of curvature of each of the respective bellows walls joints at each of the left and right ends of the cushion.

A maximum depth of the or each of the bellows is about 2 mm to about 20 mm.

A maximum depth of the or each of the bellows is about 5 mm to about 10 mm.

A maximum depth of the or each of the bellows is about 8.5 mm.

The inner bellows wall or walls is/are substantially planar.

The inner bellows wall or walls is/are substantially planar but follow a shape of the bellows along the length of the or each of the bellows.

A spacing of the inner cushion wall from the or each of the adjacent inner bellows walls is variable over a depth of the or each of the bellows.

At a first region of the cushion the spacing of the inner cushion wall from an inner bellows wall is greater than the spacing of the inner cushion wall from the same inner bellows wall at a second region of the cushion.

At each of the left and right lateral ends of the cushion the spacing of the inner cushion wall from the inner bellows wall increases from each associated bellows walls joint to the connecting wall.

The spacing of the inner cushion wall from the inner bellows wall at each of the left and right wings of the cushion is locally increased towards the connecting wall at a part of the cushion to be located adjacent an alar groove of a patient.

The inner cushion wall comprises two nasal bulges, each nasal bulge projecting medially of the cushion at a region to be located adjacent respective alar regions of a patient.

The two nasal bulges are located on respective left and right wings of the cushion and project towards each other.

The nasal bulges of the inner cushion wall are each located at a junction of the inner cushion wall with at least a proximal portion of the connecting wall of each wing of the cushion.

The nasal bulges define a rim that extends along a or the junction of the inner cushion wall with at least a respective proximal portion of the connecting wall of each wing of the cushion.

The inner cushion wall comprises a nasal aperture to receive a patient's nose.

The inner cushion wall comprises two lip bulges, each lip bulge projecting proximally of the cushion from respective portions of the inner cushion wall below the nasal aperture.

The lip bulges are spaced apart from each other across the central portion of the cushion.

The lip bulges are located on the inner cushion wall adjacent a junction of the inner cushion wall with a or the lower portion of the connecting wall.

The or each of the bellows is able to independently expand to maintain a sealing engagement of the inner cushion wall with a patient's face across a range of lateral displacements of the patient's face relative to the outer cushion wall.

The cushion is internally pressurised by a therapy airflow and the or each of the bellows expand when an external pressure resulting from a contact force of a patient's face at a bellows-adjacent portion of the inner cushion wall is less than the internal cushion air pressure.

The or each of the bellows are biased towards a collapsed or at least partially collapsed position.

The or at least one of the bellows is biased towards an expanded condition.

The or each of the bellows is biased to a partially expanded position, such that upon a lateral displacement of a patient's face relative to the outer cushion wall the bellows at one wing of the cushion is caused to expand and the bellows at the other wing of the cushion is caused to contract.

The contraction of the or each of the bellows reduces a deformation or wrinkling of the inner cushion wall upon a lateral displacement of a patient's face relative to the outer cushion wall.

A lower portion of the connecting wall has a greater thickness than an upper portion of the connecting wall.

The thickness of one or more of the bellows walls at a lower portion of the cushion are greater than the thickness of one or more of the bellows walls at an upper portion of the cushion.

The thickness of each of the bellows walls at a lower portion of the cushion are greater than the thickness of each of the bellows walls at an upper portion of the cushion.

The connecting wall includes a reinforcing rib extending across at least a portion of a width of the connecting wall between the inner cushion wall and outer cushion wall.

The or each of the bellows extends along the lower portion of the connecting wall, and the reinforcing rib or respective reinforcing ribs extend across the bellows at the lower portion of the connecting wall.

The lower portion of the connecting wall includes two reinforcing ribs, one located on the lower portion of the connecting wall on each lateral side of the nasal aperture of the inner cushion wall.

The or each reinforcing rib comprises a local thickening of the wall of cushion.

The thickening of the wall of the cushion at the or each rib extends towards an inside of the cushion.

The or each of the bellows each have a widened portion at which the spacing of the bellows walls from each other is relatively increased.

At the or each widened portion the spacing of the bellows walls at the or each bellows walls joint is increased.

At the or each widened portion the bellows walls joint comprises a flat portion.

The or each of the bellows of the cushion extend along the connecting wall around respective ends of the left and right wings of the cushion.

The or each of the bellows of the cushion extend along an upper portion of the connecting wall, and are integral with each other.

The or each of the bellows have a bellows base, and the bellows base is straight along its length in respective regions either side of the ends of each of the left and right wings of the cushion.

According to another aspect, the disclosure provides a respiratory interface comprising a cushion as herein described and two side arms to support the non-face contacting outer cushion wall of the cushion, wherein in use an expansion of the or each of the bellows comprises a movement of a respective portion of the inner cushion wall from an adjacent portion of the outer cushion wall.

An expansion of the or each of the bellows cause an associated portion of the inner cushion wall to move medially.

An expansion of the or each of the bellows cause an associated portion of the inner cushion wall to move medially and proximally.

The inner cushion wall includes an aperture for passing respiratory gases to a patient's nares.

A thickness of the inner cushion wall at a lip contacting part is increased relative to a remainder of the inner cushion wall.

A thickness of the inner cushion wall above the aperture is substantially uniform, and below the aperture a thickness of the inner cushion wall is relatively increased.

A thickness of the inner cushion wall above the aperture is about 0.3 mm.

A thickness of the outer cushion wall is about 0.5 mm.

According to another aspect, the disclosure provides a nasal cushion for a respiratory interface, the cushion comprising an inner cushion wall to contact a patient's face, a non-face contacting outer cushion wall and a first and second bellows located between the inner cushion wall and outer cushion wall,
wherein the bellows are each expandable to maintain a sealing engagement of the inner cushion wall with the patient's face under lateral movement of the patient's face relative to the outer cushion wall.

The bellows are laterally spaced apart on the cushion.

The cushion defines a central portion and two lateral wings, and the bellows are located between the inner cushion wall and outer cushion wall along at least an upper portion of each of the two lateral wings of the cushion.

The bellows extend in depth into the cushion from a most proximal part of each of the two lateral wings.

The bellows extend in depth into the cushion from a most peripheral part of each of the two lateral wings.

According to another aspect, the disclosure provides a nasal cushion for a respiratory interface, the cushion comprising an inner cushion wall to contact a patient's face, a non-face contacting outer cushion wall and a first and second bellows located between the inner cushion wall and outer cushion wall,
wherein the first bellows is expandable to maintain a sealing engagement the patient's face when the patient lies on a side of their head associated with the second bellows.

The bellows are laterally spaced apart on the cushion.

The cushion defines a central portion and two lateral wings, and the bellows are located between the inner cushion wall and outer cushion wall along at least an upper portion of each of the two lateral wings of the cushion.

The second bellows is expandable to maintain a sealing engagement with the patient's face when the patient lies on a side of their head associated with the first bellows.

According to another aspect, the disclosure provides a nasal cushion for a respiratory interface, the cushion having a central portion and left and right wings that extend to respective left and right ends from the central portion, the cushion comprising:
an inner cushion wall to contact a patient's face,
a non-face contacting outer cushion wall, and
a connecting wall that connects the inner cushion wall and outer cushion wall,
wherein the inner cushion wall comprises at least one bulge, each at least one bulge located at a junction of the inner cushion wall with the connecting wall.

Each at least one bulge comprises a projection of the inner cushion wall away from an interior of the cushion.

The at least one bulge includes two nasal bulges, one nasal bulge on the junction of the inner cushion wall with the connecting wall towards the each of the left and right ends of the cushion.

The nasal bulges extend along the junction of the inner cushion wall with the connecting wall adjacent both an upper portion of the connecting wall and a proximal portion of the connecting wall.

The nasal bulges each define a medially projecting rim of the inner cushion wall along the junction of the inner cushion wall with the connecting wall about at least part of the ends of each of the left and right wings.

The at least one bulge includes two lip bulges, the lip bulges each located either side of the central portion of the cushion at the junction of the inner cushion wall with a lower portion of the connecting wall.

The inner cushion wall comprises a nasal aperture to receive a patient's nose, and the two lip bulges each extend proximally along the junction of the inner cushion wall with the lower portion of the connecting wall from respective locations beneath the nasal aperture.

The two lip bulges each extend proximally from below a respective left and right sides of the nasal aperture.

The cushion further comprises a bellows extending in length along the connecting wall and in depth into the cushion at each of the left and right wings of the cushion.

Each of the bellows are expandable to increase a spacing between the inner cushion wall and outer cushion wall.

According to another aspect, the disclosure provides for a cushion for a respiratory mask, the cushion having an inner cushion wall to contact a patient's face and a non-face contacting outer cushion wall, and at least one bellows expandably connecting between peripheral parts of the inner and outer cushion walls.

According to another aspect, the present disclosure provides for a nasal cushion for a respiratory interface, the cushion having a central portion and left and right wings that extend to respective left and right lateral ends from the central portion, the cushion comprising:
an inner cushion wall to contact a patient's face,
a non-face contacting outer cushion wall,
a connecting wall that connects the inner cushion wall and outer cushion wall, and
a bellows, the bellows extending in length along the connecting wall and in depth into the cushion at each of the left and right wings of the cushion,
wherein the bellows is expandable to increase a spacing between the inner cushion wall and outer cushion wall.

As used herein the term “and/or” means “and” or “or”, or both.

As used herein “(s)” following a noun means the plural and/or singular forms of the noun.

For the purposes of this specification, the term “plastic” shall be construed to mean a general term for a wide range of synthetic or semisynthetic polymerization products, and generally consisting of a hydrocarbon-based polymer.

The term “comprising” as used in the specification and claims means “consisting at least in part of.” When interpreting each statement in this specification that includes the term “comprising,” features other than that or those prefaced by the term may also be present. Related terms “comprise” and “comprises” are to be interpreted in the same manner.

In this specification the directional terms “proximal” and “distal”, “lateral” and “medial”, and “upper” and “lower”, are generally to be understood in relation a patient wearing the interface, mask, or cushion, and particularly, where appropriate, are to be understood in relation to the face or specifically to the nose, of the patient.

Other aspects of the invention may become apparent from the following description which is given by way of example only and with reference to the accompanying drawings.

### BRIEF DESCRIPTION OF THE DRAWINGS

Preferred embodiments of the invention will be described by way of example only and with reference to the drawings, in which:

FIG. 1 is a view of a respiratory interface system.

FIGS. 2A and 2B are views of a cushion module for a respiratory interface.

FIG. 3 is a view of a respiratory interface system with its patient interface being worn by a model of a patient.

FIGS. 4A and 4B show two views of a patient interface in worn conditions.

FIGS. 5A-5E are each views of a cushion for a patient interface.

FIG. 6A is a sectional view of a cushion for a patient interface.

FIG. 6B is a view of the cushion of FIG. 6A with a bellows of the cushion in an expanded condition.

FIG. 6C is a view of the cushion of FIG. 6B with the bellows in a further expanded condition.

FIG. 6D is a view of the cushion of FIG. 6A with another bellows in an expanded condition.

FIG. 7A is a top view of a cushion.

FIG. 7B is a sectional view through the line BB of FIG. 7A.

FIG. 7C is a sectional view through the line CC of FIG. 7A.

FIG. 7D is a sectional view through the line DD of FIG. 7A.

FIG. 8A is view of a patient interface worn by a patient, where the patient interface has a cushion with a bellows.

FIGS. 8B and 8C are views of a patient interface in different displaced conditions on a patient's face.

FIGS. 9A and 9B are top views of a cushion with a bellows in non-expanded and expanded conditions.

FIG. 9C is a rear view of a cushion.

FIGS. 9D and 9E are, respectively, views of a cushion with a bellows in non-expanded and expanded conditions.

FIG. 10 is a side view of a cushion.

FIG. 11 is a section view through the line AA of the cushion of FIG. 9.

FIG. 12 is a section view of a cushion.

FIGS. 13A and 13B are views in opposite directions of a section taken through the height of a cushion.

FIG. 14-1 is a section view through a wing of a cushion.

FIGS. 14-2A and 14-2B are views of another embodiment of a cushion.

FIGS. 15A-D are section views of a cushion taken at different locations along the wings of the cushion.

FIGS. 16A-C are views of another embodiment of a cushion.

FIGS. 17A-C are views of another embodiment of a cushion.

FIGS. 18A-C are views of another embodiment of a cushion.

FIGS. 19A-C are views of another embodiment of a cushion.

FIG. 20-1 is a side view of a cushion illustrating the location of a bottom of a bellows.

FIG. 20-2 is a side view of another cushion.

FIG. 21 is a side view of a sectioned cushion.

FIGS. 22A-C are views of another embodiment of a cushion.

FIGS. 23A-C are views of another embodiment of a cushion.

FIG. 24 is a sectional view through a right wing of a cushion.

FIG. 25 is a sectional view through a right wing of a cushion.

FIGS. 26A-C are, respectively, a top view, rear view, and bottom view of a cushion.

FIG. 27 is a view of a cushion sectioned through its height.

FIG. 28 is a view of a cushion sectioned along its height.

FIG. 29A-C are view of another embodiment of a cushion.

FIG. 30A is a sectional view through the wing of a cushion towards the end of the wing.

FIG. 30B is a sectional view taken across the height of a cushion.

FIG. 30C is a sectional view taken across the height of a cushion, taken at a lower height than the section of FIG. 30B.

### DETAILED DESCRIPTION

Described herein are various embodiments of a cushion for a respiratory interface, and a respiratory interface including a cushion.

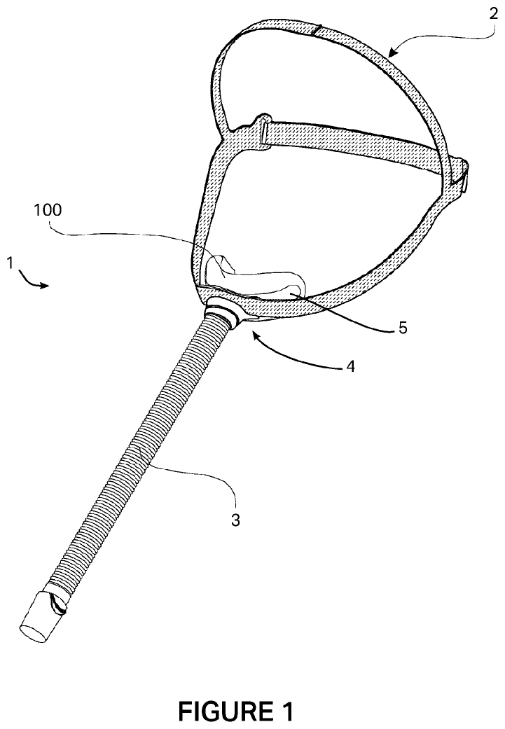

FIG. 1 is a view of an example of a respiratory interface system. The respiratory interface system includes a patient interface 1 which receives a therapy airflow from a conduit 3. The patient interface 1 provides an air path from the conduit 3 to the airways of a patient. The conduit 3 is for connection to a flow generator to provide the respiratory therapy airflow, such as a CPAP therapy, to a patient.

The patient interface 1 includes a cushion 100 and a frame 4. The cushion is for creating a seal with the face of a patient. The frame 4 connects, either directly or indirectly, to both a non-patient contacting side of the cushion 100 and the conduit 3. A headgear 2 for positioning and retaining the patient interface 1 on the face of the patient may also connect to the frame 4. The frame 4 may include two side arms 5 for engaging a side of the cushion away from the patient.

In some configurations the side arms 5 may be unitarily formed with another portion or a remainder of the frame 4, such as by injection moulding from a plastics material, for example. However, in other forms the frame 4 may have a central portion which is separate to and may be detachable from the side arms 5.

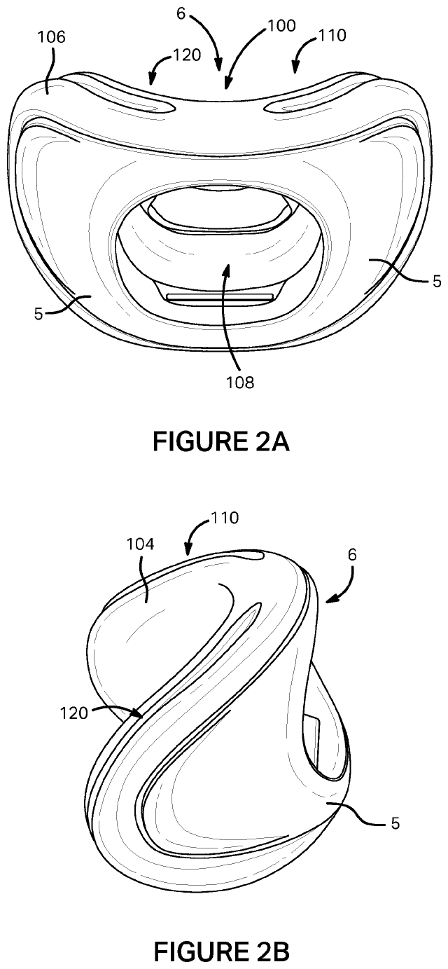

A cushion 100 may be provided as part of a cushion module 6, which includes the cushion 100 and the side arms 5. The cushion module 6 may then connect to the frame 4. FIGS. 2A and 2B are front and side views, respectively, of a cushion module 6.

A cushion 100 may be pressurised in use by a flow of therapy gases.

A cushion 100 has an inner cushion wall 104 which is for, in use, contacting the face of the patient and sealing with it. The cushion also has a non-face contacting outer cushion wall 105. A connecting wall 106 extends between the inner cushion wall 104 and the outer cushion wall 105.

The side arms 5 engage with and support the non-face contacting outer cushion wall 105. The side arms 5 may be rigid or semi-rigid and may constrain the outer cushion wall 105 of the cushion when the cushion is pressurised.

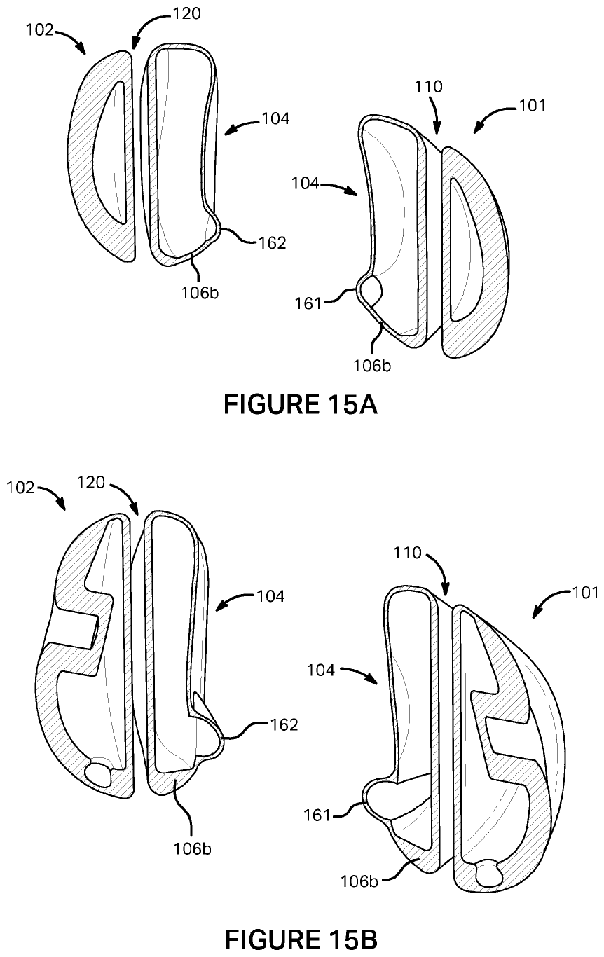

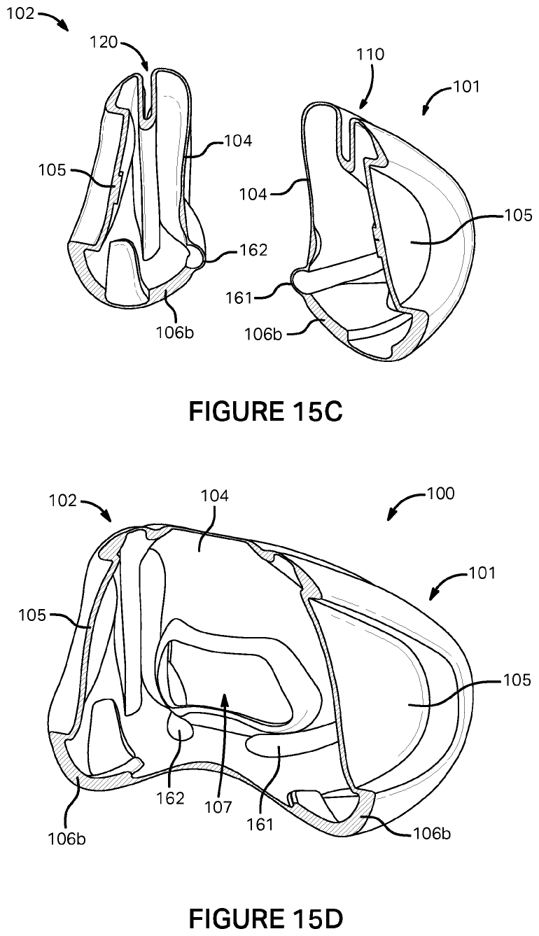

As well as constraining the outer cushion wall 105 when the cushion is pressurised, side arms 5 may also stabilise the cushion 100. To this end, the side arms 5 may be at least partially recessed within the outer cushion wall 105, or parts of the outer cushion wall may abut at least some parts of the periphery of the side arms 5. A rim or lip about the periphery of the recessed part of the outer cushion wall 105 may surround and in some configurations engage with a periphery of the side arms 5, to aid in the side arms 5 stabilising the cushion 100. The recessing of the side arms 5 into the outer cushion wall 105 may provide a more aesthetically pleasing appearance to the cushion module 6. It may also provide stability to the cushion from the relatively more rigid side arms 5. In particular, such a configuration may stabilise the cushion 100 against rolling or sliding of the inner cushion wall 104 and outer cushion wall 105 relative to each other. A recess in the outer cushion wall 105 for receiving the side arms 5 is illustrated for example in FIGS. 15A-D.

An example of this is seen in FIGS. 2A and 2B, where outer cushion wall 105 is shaped to receive the side arms 5, so that a shoulder of the outer cushion wall is provided about the periphery of the side arms 5.

The side arms 5 extend from either side of an aperture 108 in the outer cushion wall 105. From the aperture 108 the side arms 5 each extend laterally and proximally along the outer cushion wall 105. The side arms 5 may terminate at respective side arm tips which are distally located of the tips of the cushion 100. The side arms 5 may each curve towards each other along their extents away from the aperture 108 and towards the side arm tips. A height of the side arms 5 may taper along their length from the aperture 108 to the side arm tips. At least a part of the periphery of the side arms 5, and particularly parts of the periphery towards the side arm tips, may be of increased thickness to form a peripheral rim or shoulder of the side arms. Such a rim or shoulder may provide increased stiffness to the side arms 5.

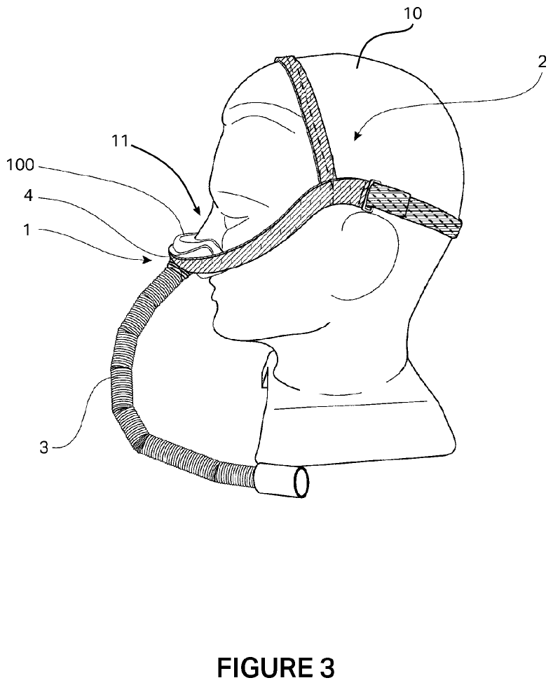

According to some embodiments the patient interface 1 may be a nasal interface, and particularly but not solely an under-nose or sub-nasal interface. As seen in FIG. 1 and in further detail in FIG. 3, the cushion 100 is provided as an under-nose cushion. The patient interface 1 is shown in FIG. 3 provided on the nose 11 of a patient. In this configuration the cushion 100 may seal around the tip of the patient's nose, at the lower left and right sides of the nose, and along the patient's upper lip or the base of their nose. The cushion may also seal along portions of the face adjacent to left and right lower flanks of the nose.

According to other embodiments the described patient interface 1 and particularly its cushion 100 may be provided as a nasal or naso-oral interface and cushion. In such embodiments the cushion 100 acts to seal around the mouth, nose, or mouth and nose as appropriate, of the patient 10.

A patient may wear a patient interface 1 while they sleep. When sleeping on their side, or when moving to their side, the interface may be pressed against a patient's face and particularly their nose, on a side of the patient's face adjacent to their bed. This may cause distortion of the cushion 100 of the interface, and result in a loss of sealing engagement of the cushion on the opposite side of the patient's face, away from their bed.

The loss of sealing contact of the cushion may interfere with the respiratory therapy. It may also cause alarm or discomfort for the patient, as pressurised therapy air may leak past the cushion at the point where sealing contact is lost. Where a patient is using the respiratory interface when sleeping, a loss of cushion seal may cause disruptions to their sleep.

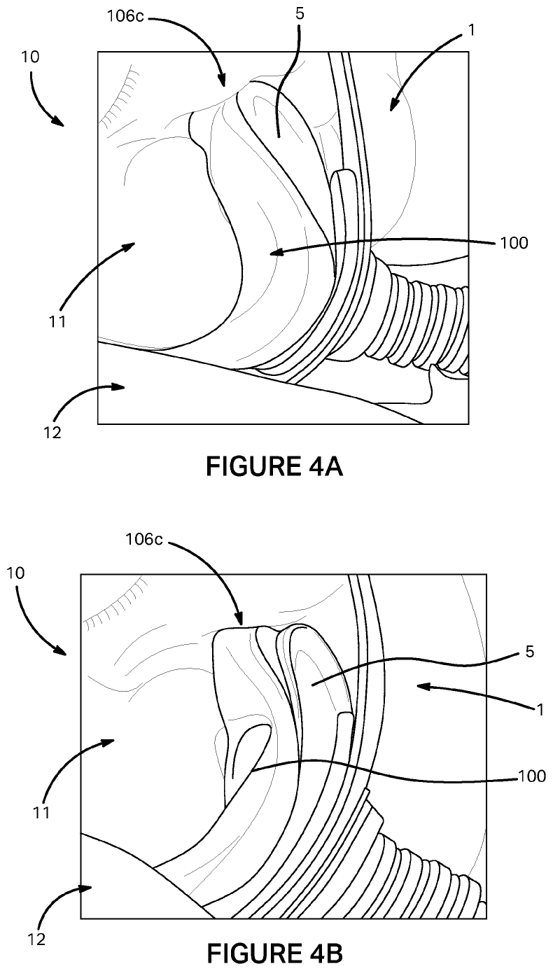

FIG. 4A is a view of part of the face of a patient 10 who is side sleeping with their head supported by a pillow or other item of bedding 12. The patient 10 is wearing a respiratory interface 1 with a cushion 100. The patient 10 is laying on the right side of their face, causing the right-hand side of the patient interface 1, from the perspective of the patient, to be displaced towards the patient's nose. This has resulted in the left-hand side of the cushion 100 losing sealing engagement with the left-hand side of the patient's nose 11.

FIG. 4B is another view of a patient 10 using a patient interface 1 resting their head on bedding 12. As in FIG. 4A, the right-hand side of the patient interface 1 has been displaced towards the patient's nose. This has resulted in distortion and wrinkling of the cushion 100 which may cause a breaking of the sealing engagement of the inner cushion wall with the left side of the patient's face.

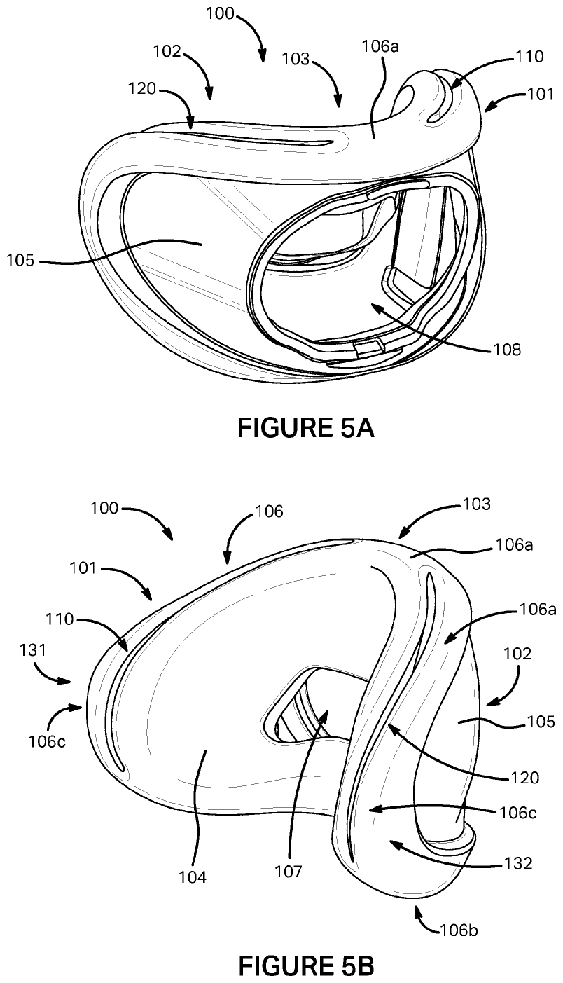

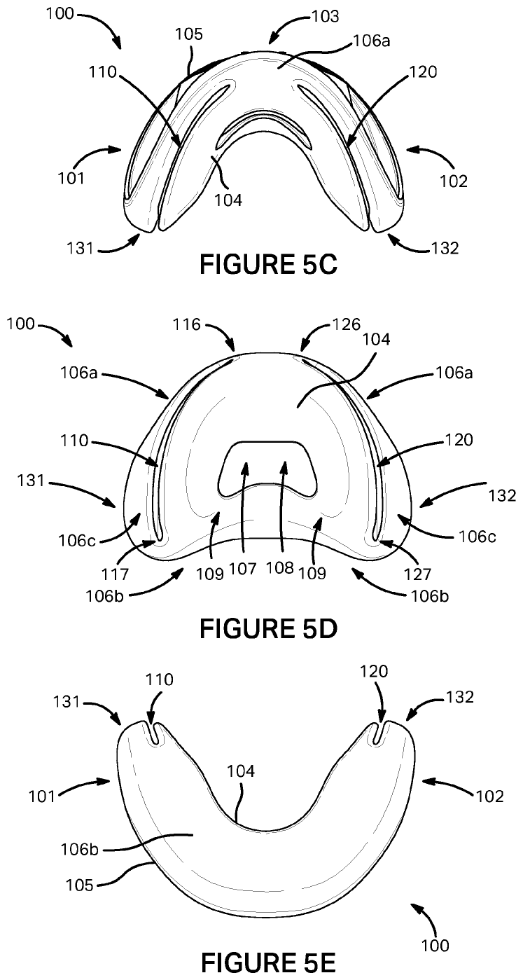

FIGS. 5A-E are various views of a cushion 100 according to one embodiment. As illustrated, the cushion 100 is a nasal cushion, specifically a sub-nasal cushion.

The cushion 100 has a central portion 103 and a respective left wing 101 and right wing 102 either side of the central portion 103. The left and right wings 101 and 102 each extend to respective left and right ends 131 and 132 of the cushion 100. The left and right wings extend rearwardly from the central portion.

The ends 131 and 132 of each wing of the cushion should be understood as respective end regions. The ends 131 and 132 of each wing of the cushion then each have respective proximal tips, being the most proximal part of each wing. In some configurations a proximal part of each of the left end 131 and right end 132 of the wings of the cushion, and more particularly respective proximal portions 106c of the connecting wall 106, are adapted when the cushion is worn to contact the patient's face adjacent to respectively left and right lower flanks of the patient's nose. In such configurations the proximal tip of each wing may be located on the proximal portion 106c of the connecting wall 106.

In addition to the proximal portions 106c of the connecting wall 106, the lower portion 106b of the connecting wall may contact the patient's face in use. In particular, the lower portion 106b may be configured to rest on the patient's upper lip in use and provide vertical support to the cushion 100 and any cushion module 6 and interface on the patient's face.

In some configurations the left and right wings may be each slightly curved when viewed from above.

The cushion 100 has a non-patient contacting outer cushion wall 105 and a patient contacting inner cushion wall 104. The inner and outer cushion walls 104 and 105 each extend along the left wing 101 from the left end 131, through the central portion 103 and to the right end 132 of the right wing 102.

The inner cushion wall 104 includes a nasal aperture 107 in the inner cushion wall 104 to receive part of the patient's nose and provide an air flow path to the patient's nares. The outer cushion wall 105 has a corresponding aperture 108 in it to allow air received from the conduit 3 to be passed through the nasal aperture 107 and to the patient's nares.

The connecting wall 106 extends between the inner cushion wall 104 and the outer cushion wall 105. In particular, the connecting wall 106 may connect the entire periphery of the inner cushion wall 104 and outer cushion wall 105 as illustrated in FIGS. 5A-E.

The connecting wall may have an upper portion 106a which connects respective upper parts of the inner cushion wall 104 and outer cushion wall 105, and a lower portion 106b which connects respective lower parts of the inner cushion wall 104 and outer cushion wall 105.

The inner cushion wall 104 and the outer cushion wall 105 may be spaced apart. The inner cushion wall 104 and the outer cushion wall 105 together with the connecting wall 106 form a hollow space which, in use, is filled with pressurised air. The cushion is configured so that when the hollow space is filled with pressurised air in use, the cushion inflates such that the inner cushion wall moves away from the outer cushion wall.

A cushion according to the disclosure may include one or more bellows. A bellows may define a fold of the cushion which, in use, may expand to aid in retaining at least a part of the inner cushion wall 104 in sealing engagement with the face of a patient. In particular, a bellows may be expandable to press a part of the inner cushion wall 104 into sealing contact with a respective side of the patient's nose.

A bellows may have a length dimension where the bellows extend along a wall of the cushion and have a depth dimension where the bellows extend into the cushion in a direction perpendicular to the associated wall of the cushion. In the length dimension a bellows extends between a first end and a second end. In the depth dimension the bellows extend from a bellows opening to a base of the bellows, being an upper surface of the bellows walls joint. The bellows may be expandable in a direction perpendicular to both their length and their depth.

A cushion including a bellows may allow for at least a degree of decoupling, in the direction of expansion of the bellows, of parts of the cushion either side of the bellows.

A bellows may be located on the connecting wall 106 of a cushion. In such a configuration the length dimension of the bellows is along the connecting wall 106 and the depth dimension of the bellows is into the cushion in a direction perpendicular to surface of the connecting wall 106.

A cushion 100 may for example include a first bellows 110 and a second bellows 120, each associated with a respective one of the two wings 101 and 102 of the cushion. FIGS. 5A-E illustrate a cushion 100 which includes a first bellows 110 and a second bellows 120.

A first bellows 110 and second bellows 120 may be provided on each of the left wing 101 and right wing 102 of the cushion at the connecting wall 106. Each of the first bellows 110 and second bellows 120 extend in length between respective ends, being a first end 116 and second end 117 of the first bellows 110, and a first end 126 and second end 127 of the second bellows 120.

A first bellows 110 and second bellows 120 may be located respectively on the left and right wings of the cushion at the respective proximal portions 106c of the connecting wall 106. At the proximal portions 106c, the bellows 110 and 120 have a depth that extends into the cushion in a distal direction. Accordingly, such a first bellows 110 and second bellows 120 may be said to extend into the cushion from the respective left and right ends 131 and 132 of the cushion.

The first bellows 110 and second bellows 120 may each extend in depth into the cushion at the end region of each wing. More particularly, the bellows may extend in depth into the cushion at the proximal tips of each wing.

In some configurations, the first bellows 110 and second bellows 120 may each have a depth that extends into each respective wing from locations either lateral or medial of the proximal tips of each wing. For example, as illustrated in FIG. 5C, the bellows 110 and 120 are each shown extending distally into the cushion 100 from locations medial of the proximal tips at the ends 131 and 132 of each wing.

In addition to or alternatively to being provided on respective proximal portions 106c, a first bellows 110 and second bellows 120 may be provided on respective upper portions 106a and/or lower portions 106b of the connecting wall 106. For example, in some configurations each of a first bellows 110 and second bellows 120 may extend sufficiently far below the ends 131 and 132 of the cushion and optionally along the lower portion 106b of the connecting wall such that in use the lower end of each of the bellows may be located adjacent to or below and/or distally of a respective alar base of each side of the patient's nose.

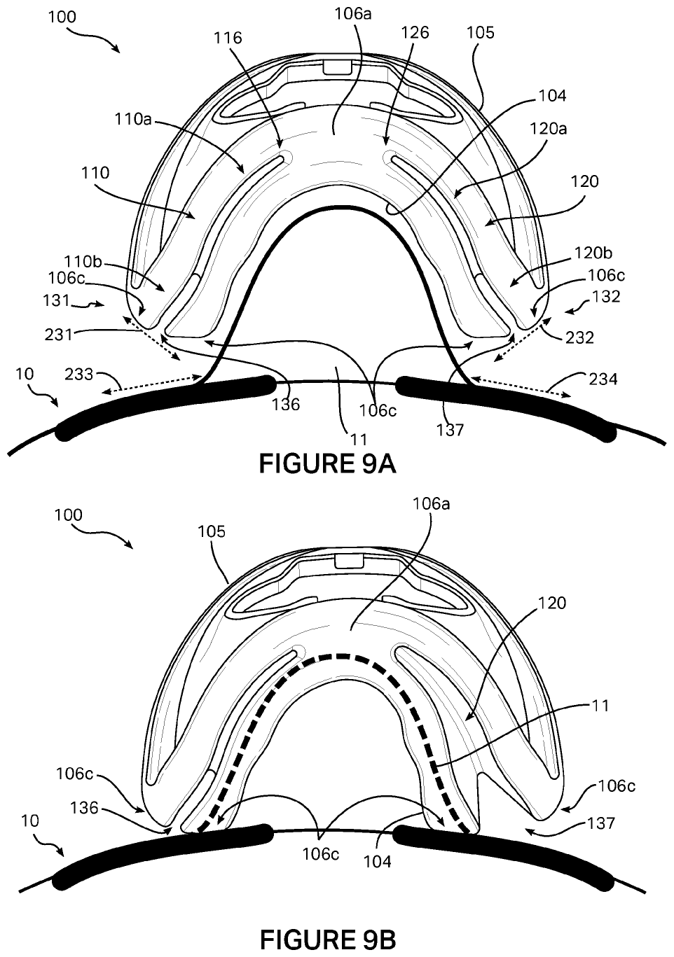

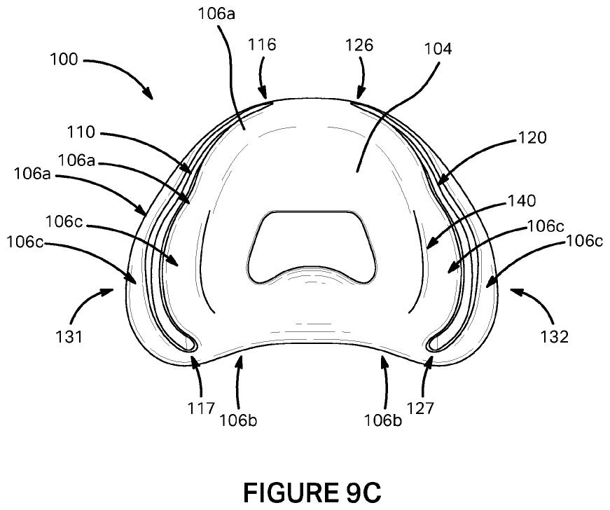

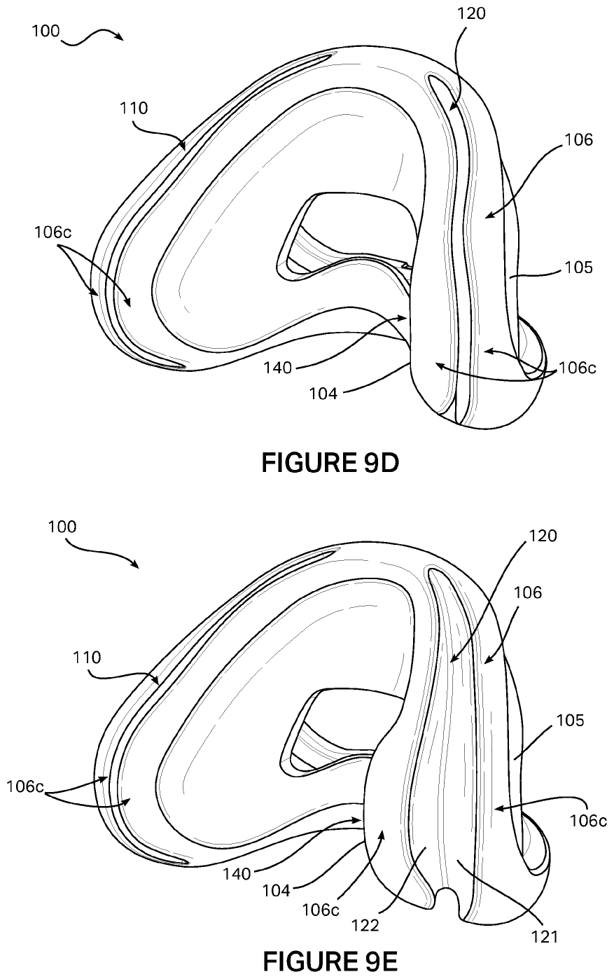

FIGS. 5A-E illustrate a configuration of a cushion 100 where a first bellows 110 and second bellows 120 are provided on both respective proximal portions 106c and respective upper portions 106a of the connecting wall 106. First ends 116 and 126 of the bellows 110 and 120 are located on the upper portion 106a of the connecting wall, and the bellows extend along their length to respective second ends 117 and 127 below the ends 131 and 132 of the cushion. While the second ends 117 and 127 of the bellows are located on the lower portion 106b of the connecting wall the bellows 110 and 120 of the configuration of FIGS. 9A-E do not extend along the lower portion 106b.

As a further example, FIGS. 23A-C illustrate a configuration of a cushion 100 where a first bellows 110 and second bellows 120 are each provided on respective upper portions 106a, proximal portions 106c, and lower portions 106b of the connecting wall 106.

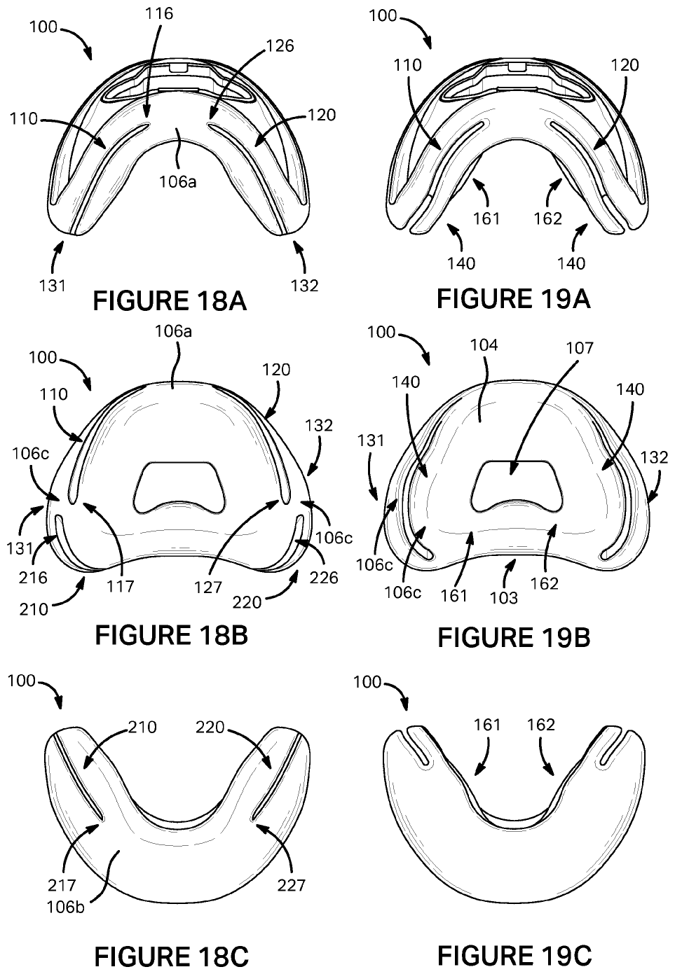

As a still further example, FIGS. 18A-C illustrate a configuration of a cushion 100 where a first bellows 110 and second bellows 120 are provided on each of the respective upper portions 106a and proximal portions 106c of the connecting wall 106 at each of the left and right wings of the cushion. The cushion 100 of FIGS. 18A-C also has a third bellows 210 and fourth bellows 220 each associated respectively with the left wing 101 and right wing 102 of the cushion, and which are provided on the lower portion 106b and proximal portions 106c of the connecting wall. The third bellows 210 and fourth bellows 220 each extend between first ends 216 and 226 and second ends 217 and 227, respectively.

While each of the bellows 110, 120, 210, and 220 are provided on the respective proximal portion 106c of the connecting wall 106, in the configuration of FIGS. 18A-C they do not extend wholly across a vertical extent of the respective proximal portions 106c and instead a proximal end of each of the bellows, being the ends 117, 127, 216, and 226, are located at or towards the respective left end 131 and right end 132 of the cushion 100.

By the use of one or more bellows provided on and extending along the connecting wall 106 a cushion may be provided which may maintain a seal between the inner cushion wall 104 and the patient's face when there is displacement, and particularly lateral displacement, of the outer cushion wall 105 or part thereof and the patient's face relative to each other.

In at least some configurations the first ends 116 and 126 of a first bellows 110 and second bellows 120 respectively may be located on the connecting wall 106 at locations on each wing above the nasal aperture 107 of the inner cushion wall 104. More particularly, in some forms the first ends 116 and 126 may be located on the connecting wall 106 above respective left and right sides of the nasal aperture 107.

Where a bellows extends in length along either or the upper portion 106a or the lower portion 106b of the connecting wall 106, a distal end of the bellows, such as the first ends 116 of the first bellows 110 of FIG. 5C, is located towards the central portion 103 of the cushion.

A bellows may follow a curvature of a respective wing of the cushion on which it is provided. A bellows may follow the curvature of the inner and/or outer cushion walls of the wing with which it is associated.

As seen in FIG. 5C a first bellows 110 and second bellows 120 each extend in depth into the cushion between their respective first ends 116 and 126 and second ends 117 and 127. The bellows 110 and 120 each have a depth that extends into the cushion in a distal direction at the respective left and right ends 131 and 132 of the cushion. More particularly, the bellows 110 and 120 each extend in depth into the cushion from the left and right ends 131 and 132 of the cushion in both distal and medial directions.

The bellows 110 and 120 curve towards each other as they extend along the wings of the cushion away from the left and right ends of the cushion and towards their respective first ends 116 and 126.

While shown in the top view of the embodiment of FIG. 5C as curving medially from the cushion ends 131 and 132 towards the central portion 103, the length of bellows may include multiple differently curved or oriented regions along the connecting wall 106.

In FIG. 5C an upper portion 106a of the connecting wall 106 is illustrated connecting between the inner cushion wall 104 and outer cushion wall 105. Also shown are the first bellows 110 and second bellows 120 respectively extending in length along the left wing 101 and right wing 102 of the cushion 100.

In the rear view of FIG. 5D the air flow path through the cushion is illustrated through both the nasal aperture 107 and the outer cushion wall aperture 108. Also seen in FIG. 5D are each of the upper portion 106a, proximal portion 106c, and lower portion 106b of the connecting wall 106.

In use, a patient's upper lip is located beneath the nasal aperture 107 of the inner cushion wall 104. A portion of the inner cushion wall 104 for contacting the upper lip of a patient in use is illustrated in FIG. 5D as extending between the arrows 109 on the inner cushion wall 104.

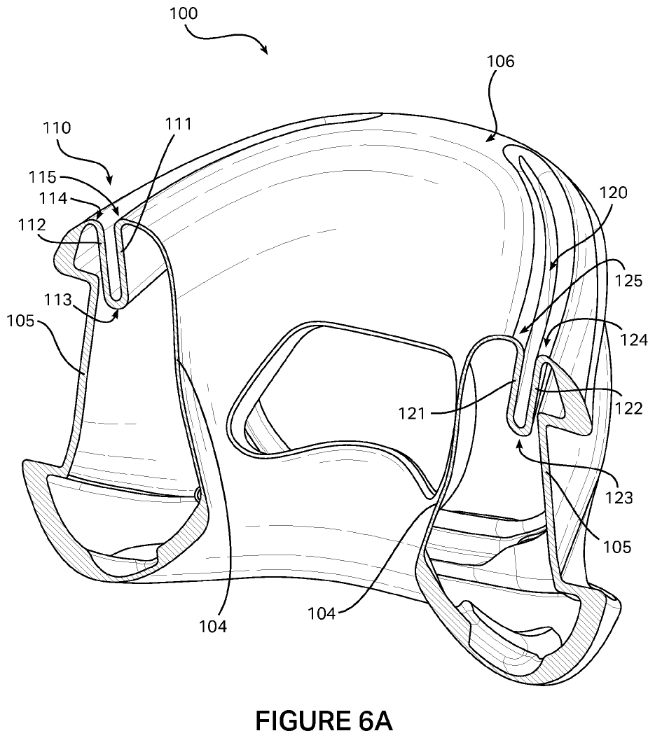

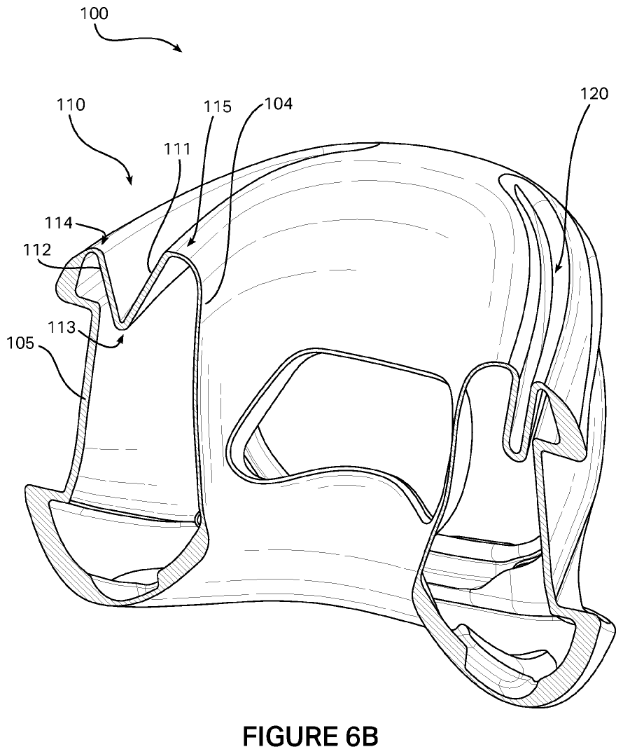

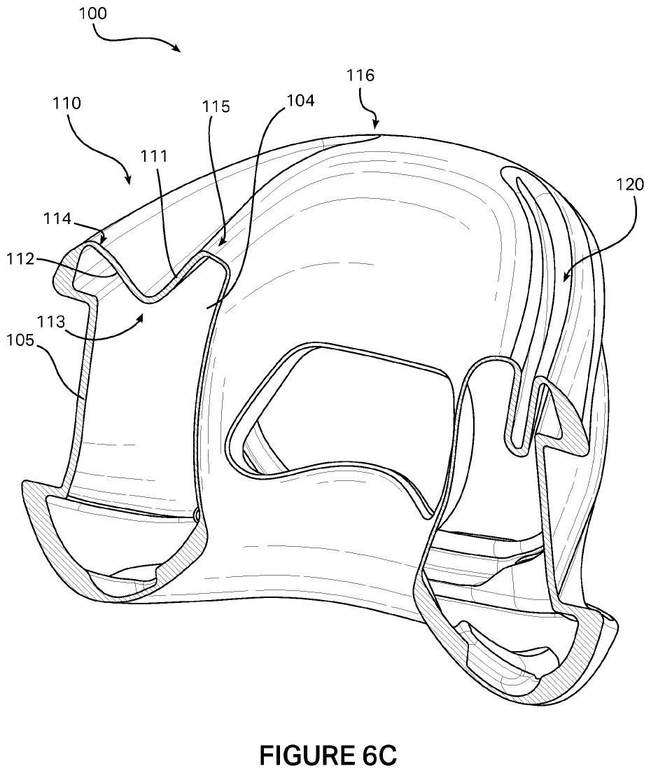

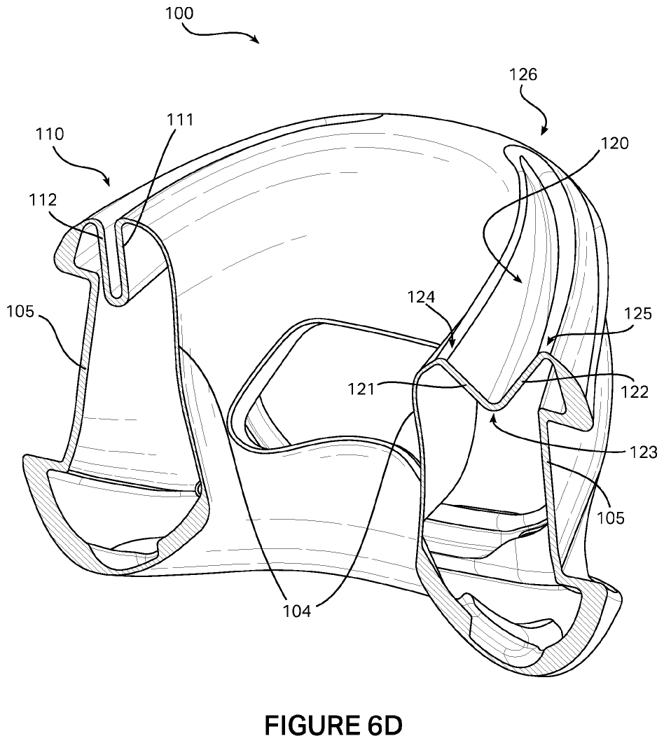

FIGS. 6A-D are each perspective views of a partially sectioned cushion 100, viewed from its patient-facing back side.

The first bellows 110 has an inner bellows wall 111 adjacent to the inner cushion wall 104, and an outer bellows wall 112 adjacent to the outer cushion wall 112. Each of the bellows walls 111 and 112 extend at a first end from the connecting wall 106, and at the other end are connected at a bellows walls joint 113. Corresponding upper joints 115 and 114 are provided between the inner and outer bellows walls 111 and 112 and the connecting wall 106.

The inner and outer bellows walls 111 and 112 are able to rotate relative to each other about the bellows walls joint 113 to allow at least an adjacent part of the inner cushion wall 104 and outer cushion wall 105 to increase in their separation from each other.

Similarly, the second bellows 120 has an inner bellows wall 121 and an outer bellows wall 122, which are connected together at a bellows walls joint 123 and connect to the connecting wall 106 at respective upper joints 125 and 124.

The inner bellows walls 111 and 121 of the first bellows 110 and second bellows 120 may each be substantially planar in form. For example, the first bellows walls 111 and 121 may each be substantially planar, but follow a curvature or other shape of the respective bellows along their length.

As illustrated in FIG. 6A, the bellows walls 111 and 112, and 121 and 122, may each be of substantially similar dimensions between their respective bellows walls joints 113 and 123, and their upper joints 115 and 114, and 125 and 124 with the connecting wall.

In various configurations, such as illustrated in FIG. 6A, the bellows walls joints 113 and 123, and their upper joints 115 and 114, and 125 and 124 with the connecting wall 106 may define curved extensions of each of the respective connected wall parts. In this configuration the joints are a curved portion that extends between the respective bellows walls or a bellows wall and a cushion wall.

One or more of the joints may have a continuous curve in cross-section. The radius of curvature of a joint having a continuously curved cross-section may vary along the cross-section. In other forms, the cross-section of a joint may define a continuous or substantially continuous radius of curvature between the two parts which it connects, at least in one condition of the bellows. For example, the radius of curvature of a joint may be substantially continuous when the bellows are in a closed condition, but discontinuous when the bellows are in an open condition.

In other configurations one or more joints may be discontinuously curved. For example, a joint may include one or more non-curved portions, such as a flat base between two lateral curved portions.

In some configurations a bellows walls joint, or at least some portions of the joint, are relatively more flexible than the parts they connect. For example, a bellows walls joint may be relatively more flexible than the bellows walls that it connects. Or, an upper bellows joint may be relatively more flexible than the one bellows wall and part of the connecting wall which it joins.

Where the cushion is formed from a single material, a bellows walls joint may be thinner than the components it connects in order to provide locally increased flexibility at the joint, or part thereof.

A bellows walls joint may be biased towards a particular condition. For example, the bellows may be biased to or towards a closed condition. In other configurations, a bellows may be biased towards a neutral condition, being a condition between a closed and an open condition. In still other configurations, a bellows may be biased to or towards an open condition.

Accordingly, following use of the cushion, the bellows may return to or towards a desired at-rest condition.

Similarly, during use of the cushion which results in expansion or contraction of the bellows, a bias of one or more of the bellows walls joints may provide a threshold of pressure within the cushion and/or pressure on the inner cushion wall and outer cushion wall of a wing of the cushion for which the bellows or bellows part will begin to expand or contract.

During therapy, pressure within the cushion may force the cushion to inflate and the bellows to expand, moving the inner cushion wall 104 medially towards the patient's nose until the patient's nose exerts a sufficient counteracting force. Accordingly, when the cushion is pressurised, the bellows may expand and contract responsive to movement of the interface 1 and the patient's face relative to each other and allow the sealing engagement of the inner cushion wall with the patient's nose to be maintained during such relative movement.

In some forms, one or the other of an inner bellows wall 111 or 121 and an outer bellows wall 112 or 122 may be taller than the other between their respective bellows walls joint and respective upper joints with the connecting wall. For example, an inner bellows wall 111 may be taller than the outer bellows wall 112, such that when the bellows is collapsed the upper joint 115 of the inner bellows wall 111 with the connecting wall sits higher than the upper joint 114 of the outer bellows wall 112 with the connecting wall. A bellows may be configured in this manner along its entire length, or only along particular regions. For example, the bellows may have an inner bellows wall which is taller than the adjacent outer bellows wall at the proximal portion 106c of the connecting wall which may contact the face of the patient in use. This will result in the inner bellows wall projecting more proximally than the outer bellows wall at the proximal portion 106c.

Such a configuration may aid in preventing contact of the patient's face with the proximal portion 106c on both sides of the proximal openings 136 and 137 of the bellows. Where the patient's face contacts the proximal portion 106c of the connecting wall on both sides of the proximal openings 136 and 137 of the bellows, the expansion or contraction of the bellows may cause stretching or pinching of the patient's face between the two sides of the bellows. Accordingly, such a configuration where one of the bellows walls and particularly the inner bellows wall projects relatively more proximally than the other bellows wall at least at the proximal portion 106c may be desirable for ensuring the bellows are able to expand and contract without being inhibited by the patient's face, and in preventing discomfort for the patient.

The bellows walls joints, such as the wall joints 113 and 123 seen in FIGS. 6A-D, may be of the same thickness and/or stiffness as the bellows walls 111 and 112, and 121 and 122, respectively. Similarly, the upper joints 114 and 115, and 124 and 125, of the bellows walls with the connecting wall 106 may be of the same thickness and/or stiffness.

In use it may be desirable to prevent a blow-out of the bellows, whereby the bellows are inflated so that the fold of the bellows is inverted, and the bellows walls joint protrudes upwardly of the upper joints with the connecting wall. Blow-out may occur due to the pressure within the cushion acting on the bellows. Blow-out may cause distortion of the cushion and a resulting loss of sealing engagement with the patient's face.

To prevent blow-out of the bellows one or both of the bellows walls joints, such as the bellows walls joints 113 and 123 of FIG. 6A, and the joints with the connecting wall, such as the upper joints 114 and 115, and 124 and 125 of FIG. 6A, may be stiffened. This may be provided through either a local stiffening of the cushion material, or simply by a local increase in the thickness of the cushion.

In some configurations a thickness of each of the bellows walls is greater than a thickness of the inner cushion wall but is less than a thickness of the outer cushion wall.

In some configurations, the bellows walls may have a thickness of about 0.5 mm.

Either or both of the joints of the bellows walls with each other or their joints with the connecting wall may have a greater thickness than a thickness of the bellows walls. For example, where the bellows walls have a thickness of about 0.5 mm, the joints of the bellows walls with each other may have a thickness of about 0.7 mm to about 1.0 mm.

One or more of the bellows walls joints and upper joints with the connecting wall may biased to a particular condition, such as will, when the cushion is at rest, either retain the bellows in or towards a contracted condition or in or towards an expanded condition.

In at least some preferred forms the bellows of a cushion are biased to their contracted condition, such that the bellows are partially or wholly contracted when the cushion is at rest. A strength of any bias of the bellows may be configured to be strong enough that when the cushion is not inflated the bellows will be caused to return to their contracted condition, but not so strong as to inhibit the expansion of the bellows under a normal pressurisation condition of the cushion.

In some configurations the walls of each bellows, for example the bellows walls 111 and 112 of the first bellows 110, extend substantially parallel to each other yet are spaced apart when the cushion is at rest. In other configurations, the bellows walls when the cushion is at rest may not be spaced apart from each other, or be angled together towards their upper extents, such that when the bellows are collapsed the bellows present minimal or no opening of the bellows fold to the patient.

The bellows of a cushion has a depth defined between the upper joints with the connecting wall and the bellows walls joint of the inner and outer bellows walls.

In some configurations the depth of one or both bellows 110 and 120 decreases towards each of their respective ends. However, in other embodiments, the depth of the bellows may decrease towards only one of their respective ends, or each bellows may have a depth that is constant between its respective ends, or its depth may vary in other ways over its length.

The depth of a bellows will determine an amount of expansion that the bellows can provide. Accordingly, where bellows vary in depth between their two ends, the bellows may provide for different amounts of possible expansion of the bellows and consequently different amounts of medial deflection of the inner cushion wall at different locations between their two ends.

The depth of a bellows may be customised along its length between its first and second ends to provide expansion at locations of the patient's face which are most likely to lose sealing engagement with the cushion. For example, in some configurations the inner cushion wall may be prone to a loss of seal with the patient's face around the alar region and particularly at or around the alar facial grooves of the patient's nose. Accordingly, where a bellows is present at this region it may be provided with a relatively greater depth than at other regions of the bellows, to allow for a relatively greater amount of movement of the inner cushion wall to aid in maintaining sealing engagement at that part of the patient's face.

Customising the depth of the bellows along their length to provide only the amount of expansion that is desired in use at individual regions or points along the bellows may also increase the stability of the cushion. Relatively shallower parts of a bellows may have greater resistance to rolling of the inner cushion wall and outer cushion wall relative to each other than relatively deeper parts of a bellows. Similarly, shallower parts of a bellows may be less prone to blow-out of the bellows than are deeper parts of the bellows.

When the cushion 100 is pressurised and as a result the bellows will be caused to expand unless or until the patient's face exerts a sufficient counteracting force on the inner seal wall 104. The expansion of the bellows may occur along both wings of the cushion at once, for example if the patient has a small nose or if the cushion is configured so that the bellows are to expand under normal correctly positioned use of the patient interface by a patient. More particularly however, one bellows may expand at a time, responsive to a respective displacement of the interface on the patient's face such as may occur during side sleeping.

For example, where the first bellows 110 expands, the bellows walls 111 and 112 are caused to pivot relative to each other about the bellows walls joint 113. The expansion of the bellows 110 results in an increased separation of the internal cushion wall 104 and the external cushion wall 105. The condition of a cushion 100 where the first bellows 110 has partially expanded is illustrated in FIG. 6B.

Where a cushion module 6 includes two side arms 5, or the movement of the outer cushion wall 105 is otherwise constrained by two side arms 5, the expansion of a bellows such as the bellows 110 of FIG. 6B may be caused to occur largely or wholly in a medial direction, where the inner cushion wall 104 is displaced medially away from the outer cushion wall 105.

According to various configurations the cushion 100 may be structured so as to direct the expansion of the bellows to occur medially, either in addition to the limitations provided by any side arms 5, or in the absence of the side arms. For example, as illustrated in FIG. 6B, the inner cushion wall 104 may be formed to be relatively thinner and/or less stiff than the outer cushion wall 105, so that the inner cushion wall 104 may more readily conform to a shape of the patient's face.

In at least some embodiments it may be desirable to maximise a degree to which the bellows expand medially, and/or to minimise a degree to which the bellows expand laterally. This may provide, for any given bellows, an increased range of displaced positions of the patient's face relative to the outer cushion wall 5 for which the cushion may maintain a sealing engagement of the inner cushion wall 104 with the patient's face.

FIG. 6C illustrates the cushion 100 of FIG. 6B, but with the bellows 110 further expanded.

As seen in FIG. 6C the bellows 110 are of increasing depth away from the first end 116 of the bellows 110. The resulting variation in the amount of displacement caused by the expansion of the bellows is seen in FIG. 6C, where the amount of displacement decreases from where the section has been taken through the bellows towards the first end 116 of the bellows 110.

A maximum degree of expansion of a bellows may be determined by both the depth of the bellows and the amount of rotation permitted at one or more of the bellows walls joints of its walls.

As seen in the expanded condition of FIG. 6C, the walls 111 and 112 of the bellows 110, at least at the sectioned part, define an angle of approximately 90 degrees. Various configurations of a cushion may provide for relatively greater degrees of expansion of one or more of their associated bellows, for example so that at a maximum expansion of the bellows part or all of the bellows walls define an angle of greater than 90 degrees, and even of approximately 180 degrees, between them.

FIG. 6D illustrates the cushion of FIG. 6A, but where the second bellows 120 is partially expanded while the first bellows 110 remains contracted. As seen in FIG. 6D the inner bellows wall 121 and outer bellows wall 122 have hinged away from each other about their bellows walls joint 123, medially displacing the inner cushion wall 104 of the right wing of the cushion 100.

Where the configuration of FIGS. 6B and 6C are such as could result, for example, from a displacement of the interface 1 or particularly the outer cushion wall 5 to the left of the patient's face, the configuration of FIG. 6D is such as could result from the displacement of the interface or outer cushion wall 5 to the right of the patient's face.

As illustrated in FIG. 6D the bellows 120 also decrease in depth towards their first end 126.

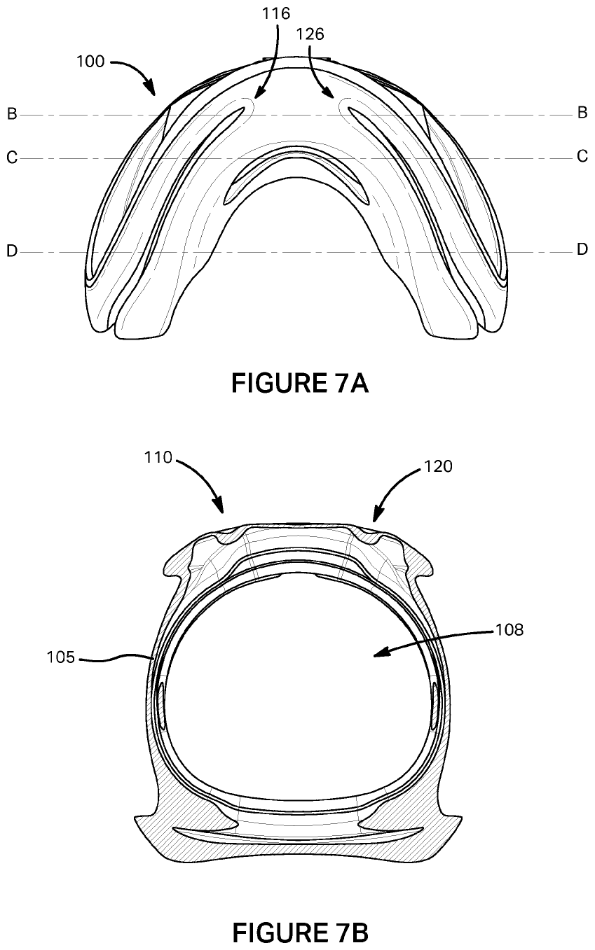

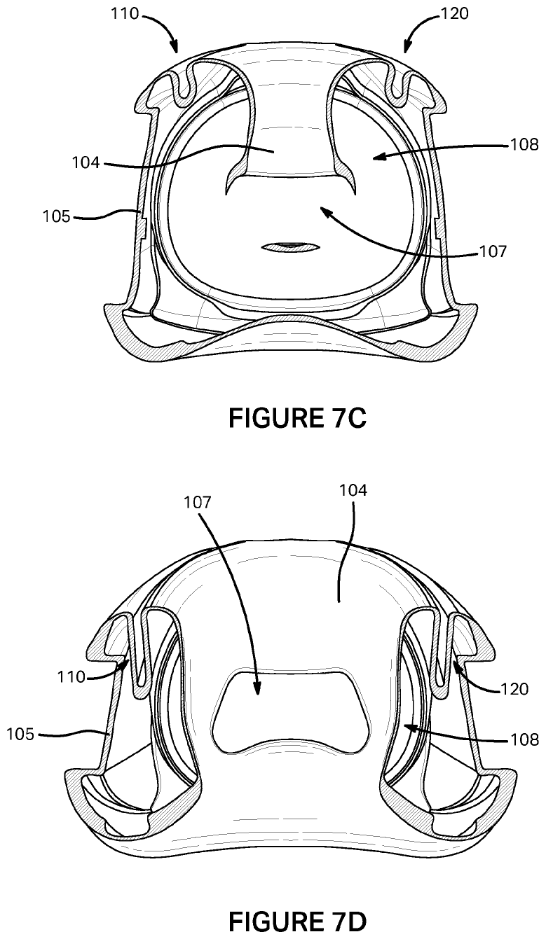

FIG. 7A is a top view of a cushion 100 according to an embodiment.

FIG. 7B is a sectional view vertically through the cushion at line BB of FIG. 7A, viewed from the rear of the cushion towards its front. The line BB passes through the cushion just proximally of the first ends 116 and 126 of the bellows 110 and 120, and just distally of the aperture 108 in the outer cushion wall 105. In FIG. 7B the bellows 110 and 120 have each begun but are of a depth only approximately equal to the thickness of the bellows walls.

FIG. 7C is another sectional view more proximally than that of FIG. 7B, taken vertically through the cushion along the line CC of FIG. 7A. The line CC passes through the cushion at a distal part of the nasal aperture 107. As seen in FIG. 7C the depth of the bellows 110 and 120 has gradually increased relative to the depth of the bellows in FIG. 7B and the base of the bellows each align with respective top shoulders of the outer cushion wall 105 where it engages side arms 5.

At the location of the section of FIG. 7C the depth of each of the bellows is about 4 to about 6 times the thickness of the bellows walls.

Finally, FIG. 7D is a still more proximate sectional view taken vertically through the cushion at the line DD of FIG. 7A. The line DD is located just distally of the extent to which the bellows extend in depth into the proximal portion 106c of the connecting wall 106. The line DD is located at the point of a change of direction of the bellows along the upper portion 106a of the connecting wall. More proximally of the line DD the bellows are oriented on the connecting wall relatively more laterally than they are distally of the line DD. As seen in FIG. 7D the depth of the bellows 110 and 120 are relatively greater than that of either of the sections of FIG. 7B or 7C, and the base of the bellows are each located below the respective top shoulders of the outer cushion wall 105 where it engages side arms 5.

At the location of the section of FIG. 7C the bellows may have a depth of about 8 to about 14 times the thickness of the bellows walls.

The depth of a bellows may be configured along the length of the bellows to enable the desired degree of movement of an associated part of the inner cushion wall 104 relative to the outer cushion wall 105.

In some configurations the depth of a bellows at or around an associated proximal portion 106c of the connecting wall may be about 1/10^{th} to about ¼ of the lateral distance between the two proximal tips of the cushion.

According to various configurations the bellows of a cushion may have a maximum depth of, for example, about 15 mm.

In other configurations the maximum depth of the bellows in at least some locations may be about 9 mm.

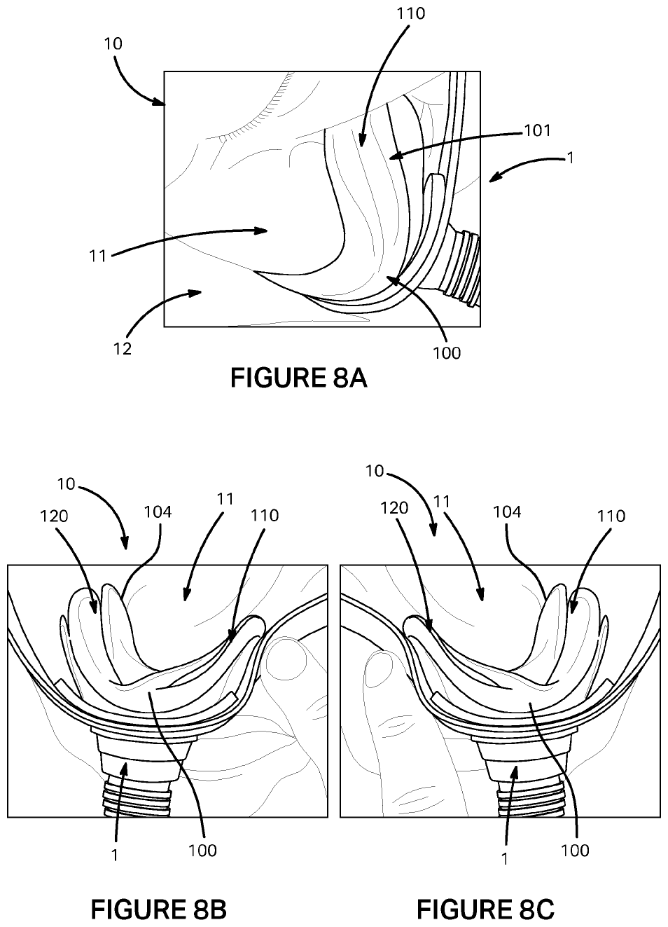

FIG. 8A to 8C are views of various use conditions of a patient interface 1 having a cushion with two bellows 110 and 120, one on each respective wing of the cushion.

In FIG. 8A, as in FIGS. 4A and 4B, the patient 10 is shown resting their face and particularly nose 11 on some bedding 12, so that the patient interface 1 is forced to the left side of their face. As seen in FIG. 8A, a resulting reduction in contact force from the patient's face along the left wing 101 has allowed the first bellows 110 to expand under the pressure of the therapy airflow within the cushion. The expanded first bellows 110 may allow the inner cushion wall to maintain a sealing engagement with the left side of the patient's face.

Similarly, FIGS. 8B and 8C show front-on views of a patient 10 with the patient interface displaced either side of their face, such as may occur during side-sleeping. In these examples, the nose 11 of the patient 10 is twisted. Such twisting of the nose of the patient may change the geometry around which the inner cushion wall 104 is required to seal. In particular, under such twisting the alar facial groove on the side of the patient's face where their nose is twisted towards may be deepened, and/or the curve around the side of the patient's nose to the alar groove and alar base be made more acute by the laterally projecting tip of the patient's nose. These characteristics may increase the chance of the inner cushion wall not being able to seal with the patient's face. An increased depth of the bellows in a region adjacent to the alar region and particularly the alar facial groove may be utilised to attempt to combat these effects.

As seen in FIG. 8B, the interface 1 has been displaced to the right of the patient's face as may be the case when the patient lies on their left-hand side, allowing the second bellows 120 to expand and fill the space on the right side of the patient's nose 11 that has resulted from the displacement of the interface.

In FIG. 8C the interface 1 has been displaced in the opposite direction to that of FIG. 8B, towards the left side of the patient's face as may be the case when the patient lies on their right-hand side. In this condition the first bellows 110 has expanded to fill the space at the left side of the patient's nose 11 that has resulted from the displacement of the interface.

As previously described, a cushion 100 may have proximal portions 106c of the connecting wall which when the cushion is worn by a user, are most proximate to the patient and contact the patient's face. Also as previously described, where the bellows of a cushion extend into the cushion from the ends 131 and 132 of the cushion's wings, respective proximal openings 136 and 137 of the bellows may be located at the proximal portion 106c which may contact the patient's face. Interaction of the patient's face with the bellows may impede the performance of the bellows or may additionally or alternatively cause discomfort for the patient such as by pinching or stretching of their skin upon contraction and expansion of the bellows.

Accordingly, it may be desirable to minimise or eliminate the potential for interaction of the patient's face with the proximal openings 136 and 137 of the bellows. In some configurations this may be provided as previously described by differently sized inner and outer bellows walls, to locate one side of the bellows at the proximal openings 136 and 137 more distally of the other, to reduce the potential for the patient's face to contact both sides of each bellows opening.

In other configurations the bellows may be additionally or alternatively shaped or oriented along the wings so as to locate the proximal openings 136 and 137 of the bellows so that they are less likely to engage with the patient's face.

For example, the bellows or at least a proximal portion of the bellows may be configured so as to locate the proximal openings 136 and 137 of the bellows towards the lateral sides of each wing, and particularly laterally on or of the proximal portions 106c of the connecting wall 106. In use the engagement of a patient's face may be primarily at respective medial sides of the proximal portion 106c of each wing. Accordingly, by locating the proximal openings 136 and 137 of the bellows on lateral parts of, or laterally of, the proximal portions 106c, the bellows openings may be located away from the patient's face or at least any engagement of the bellows openings with the patient's face may be reduced.

Where the bellows openings 136 and 137 are located laterally on or of the proximal portion 106c of the connecting wall, the bellows may extend in depth from the openings into the cushion at any given angle. However, according to various configurations, it may be desirable that the direction the bellows extend into the cushion from each opening 136 and 137 is such that they are non-perpendicular to the face of the patient in use. Such a configuration may mean that the direction of extension of the bellows into the cushion from each respective bellows opening 136 and 137 is non-parallel to a longitudinal direction of each wing of the cushion towards its respective end 131 and 132.

Where the bellows extend in depth into the cushion so that they are in-use non-perpendicular to the face of the patient, an amount of the expansion and contraction of the bellows which occurs along the patient's face may be reduced, and accordingly any stretching or pinching due to contact of the patient's face across the proximal openings 136 and 137 of the bellows may be reduced.

FIG. 9A is a top view of an embodiment of a cushion 100 where the bellows each have two differently oriented regions, and the depth of the bellows from the openings 136 and 137 extend in a direction non-perpendicular to the face of the patient. In FIG. 9A the cushion 100 illustrated next to part of the face and nose 11 of a patient. The first bellows 110 has a first region 110a between its first end 116 and the point where the bellows 110 extends into the end 131 of the left wing 101 of the cushion from the proximal portion 106c of the connecting wall. The first bellows 110 has a second region 110b that continues from this point to the left end 131 of the cushion.

The first region 110a follows a curved shape of the left wing of the cushion, extending in length proximally and laterally from the first end 116 of the bellows 110.

At the first region 110a the first bellows 110 may also be curved from the first end 116 of the bellows in a medial direction.

At the location where the bellows extend in depth into the cushion from the proximal portion 106c of the connecting wall, the longitudinal direction of the bellows along the upper portion 106a of the connecting wall changes, and in the second region 110b the bellows opening 136 is located towards a lateral portion of the wing 101.

At the first region 110a of the length of the bellows 110 the bellows curves medially, while from the depth extent of the bellows 110 into the left wing 101 from the proximal portion 106c and towards the proximal opening 136 the second region 110b of the bellows are oriented laterally outwards. This change of direction in the orientation of the bellows defines a kink in the bellows between the first region 110a and second region 110b.

The second bellows 120 has a first region 120a towards its first end 126 and a second region 120b towards the right end 132 of the cushion which correspond to the first and second regions 110a and 110b of the first bellows 110.

The changed orientation of the second regions 110b and 120b of the first bellows 110 and second bellows 120 causes the respective proximal openings 136 and 137 of the bellows to be located more laterally at the ends 131 and 132 of the respective wings of the cushion than if the bellows had continued to their respective proximal openings 136 and 137 on the trajectory of the respective first regions 110a and 120a of each bellows. Such a configuration may also reduce the potential for the patient's face to contact the proximal portion 106c of the connecting wall across both sides of the bellows at each respective proximal opening 136 and 137 of the bellows.

For example, as seen in FIG. 9A, the lines 231 and 232 illustrate a direction of expansion of each of the bellows 110 and 120 at their proximal openings 136 and 137 of the bellows, and the lines 233 and 234 illustrate an approximate tangent of the face of the patient 10 adjacent the proximal openings 136 and 137. The laterally outwards orientation of the bellows towards the proximal openings 136 and 137 of the bellows at the second regions 110b and 120b of the bellows causes the angle between the respective lines 231 and 233, and 232 and 234 to be angled further away from each other than if the proximal openings 136 and 137 were located relatively more medially on each wing.

FIG. 9B illustrates the cushion 100 of FIG. 9A engaged on the nose 11 of a patient 10, with the second bellows 120 expanded. The inner cushion wall 104 surrounds the patient's nose 11, which is shown in FIG. 9B by a dashed line. The patient's nose 11 and the cushion 100 are displaced towards each other at the left of the patient. The proximal portions 106c of the connecting wall contact the patient's face, and the location of the proximal openings 136 and 137 of the bellows are both lateral and distal of the adjacent part of the patient's face, so that contact of the patient's face with the connecting wall on both sides of either bellows may be reduced or avoided. Due to the pressurisation of the cushion 100 the second bellows 120 has expanded medially into the right-hand side of the patient's nose 11. While not shown in FIG. 9B, a pair of side arms 5 may support the outer cushion wall 105 to direct expansion of the bellows to occur predominantly or solely medially rather than laterally.

A loss of sealing engagement of the inner cushion wall 104 of a cushion 100 may in some situations be particularly prone to occurring around the alar region of a patient's nose. For example, as illustrated in FIGS. 8B and 8C, twisting of the patient's nose such as during side sleeping may deepen or make more acute the alar region, including particularly at the alar base and alar groove. If the cushion cannot conform to these shapes, one or more gaps may be formed between the inner cushion wall and the patient's face, allowing therapy air to leak away.

In some configurations a cushion 100 may include one or more bulges of the inner cushion wall 104. At a bulge the inner cushion wall 104, when the cushion is not pressurised, projects away from the interior of the cushion. A bulge may define a local change in curvature of the seal relative to the surrounding parts of the cushion, and particularly of the inner cushion wall 104.

Bulges may be utilised to allow the inner cushion wall 104 to conform to grooves or creases of the patient's face in order to maintain a sealing engagement.

Bulges may be formed as curved protrusions of the inner cushion wall 104. Each bulge may be in the form of a hollow rib-like structure.

Bulges may additionally or alternatively be utilised to create regions of increased engagement with the patient's face. Bulges may additionally or alternatively be utilised to improve the seal with the patient's face when the contact pressure of the inner cushion wall 104 on the patient's face changes, or the inner cushion wall 104 is displaced relative to the person's face. For example, a bulge may at least partially flatten when the patient's face is normal contact with the inner cushion wall 104 but be able to expand to maintain sealing contact with the patient's face if the contact pressure of the inner cushion wall 104 with the patient's face is reduced or the inner cushion wall is displaced away from the patient's face.

According to some configurations, a cushion 100 with bulges of the inner cushion wall 104 may not include any bellows.

According to other configurations, a cushion 100 with bulges of the inner cushion wall 104 may include bellows, such as a first bellows 110 and second bellows 120.

In some configurations, nasal bulges 140 of the inner cushion wall 104 may be provided on a cushion 100 so that they are adjacent to an alar region of the patient in use. This may aid in allowing the inner cushion wall 104 to conform to the shape of folds or grooves of the alar region. Nasal bulges 140 may be provided as a rim about at least part of a junction of the inner cushion wall 104 and at least the proximal portion 106c of the connecting wall 106. The rim formed by such nasal bulges may additionally extend along at least part of a junction of the inner cushion wall 104 with either or both of the upper portion 106a and lower portion 106b of the connecting wall 106.

FIG. 9C shows a back view of the cushion 100 of FIGS. 9A and 9B. According to the embodiment illustrated in FIG. 9C, nasal bulges 140 are provided on the inner cushion wall 104 towards each end 131 and 132 of the respective cushion wings of a cushion 100 which includes a first bellows 110 and a second bellows 120. As seen in FIG. 9C the nasal bulges 140 are provided on the inner cushion wall 104 towards the ends 131 and 132 of each wing of the cushion and along a junction of the inner cushion wall 104 with the connecting wall 106. The nasal bulges 140 each project medially from the inner cushion wall 104 along the junction with the connecting wall 106.

FIGS. 9D and 9E are perspectives views of the cushion 100 of FIGS. 9A-C. The cushion 100 has respective bellows 110 and 120 associated with each of the left and right wings of the cushion. In the configuration of FIGS. 9D and 9E the bellows 110 and 120 have a depth that varies along their length. In particular, the bellows may have their greatest depth around the proximal portions 106c of the connecting wall. More particularly, the greatest depth of the bellows may be either at or above the proximal portions 106c of the connecting wall. The cushion 100 is provided with nasal bulges 140 on each side of the inner cushion wall 104 adjacent to the connecting wall 106 and towards each of the ends of the cushion.

FIG. 9D illustrates the cushion 100 with the bellows 110 and 120 in a contracted condition. FIG. 9E illustrates the cushion 100 with the second bellows 120 expanded. The different depths of the bellows 120 along its length means that different amounts of displacement of the inner cushion wall 104 are caused by the expansion of the bellows. In particular, due to the increased depth of the bellows 120 around the proximal openings of the bellows, more bellows expansion occurs here than at other parts of the bellows 120. When a cushion with a bellows having a nasal bulge 140 on its inner cushion wall expands the nasal bulge 140 may be displaced medially. This is illustrated of the nasal bulge 140 of FIG. 9E where the second bellows 120 has expanded.

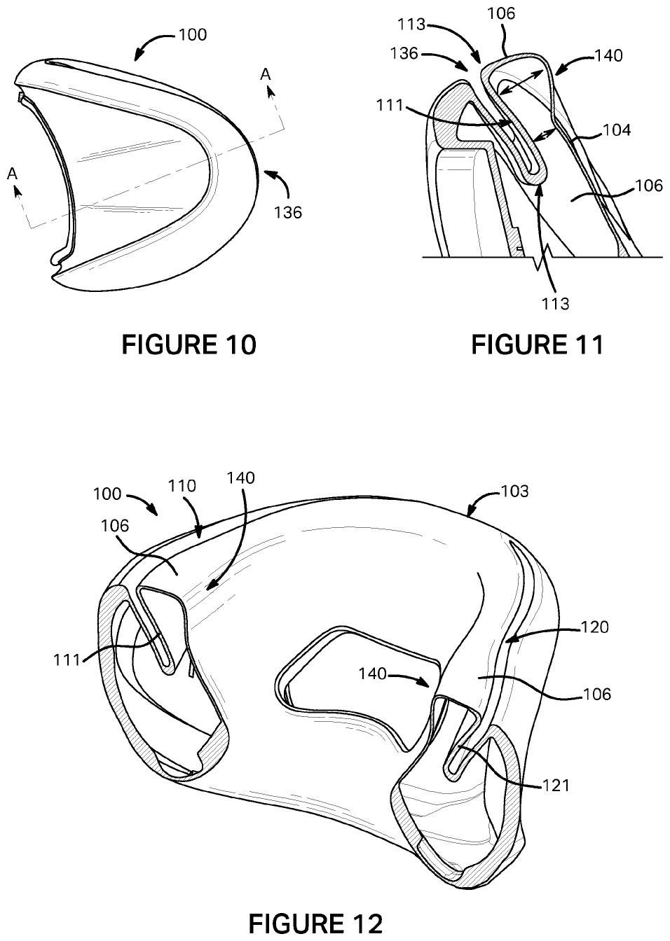

FIG. 10 is a side view of a cushion 100 which has nasal bulges 140 of its inner cushion wall 104. FIG. 11 illustrates part of a section view taken in the direction of AA through the cushion 100 of FIG. 10, showing the left wing of the cushion. The cushion 100 has a nasal bulge 140 of its inner cushion wall 104 at a part of the inner cushion wall 104 which is adjacent to the connecting wall 106 and towards the end of the wing. An inside of the connecting wall 106 is visible within the sectioned cushion of FIG. 11.

At the nasal bulge 140 the inner cushion wall 104 projects away from the interior of the cushion.

The projection causes a spacing between the inner bellows wall 111 and the inner cushion wall 104 to increase along the height of the inner bellows wall towards the upper joint 115 with the connecting wall 106. More particularly, adjacent to a middle of the height of the inner bellows wall 111 the inner cushion wall 104 inflects away from the inner bellows wall 111 to define the nasal bulge 140.

A nasal bulge 140 such as is illustrated in FIG. 11 may be provided on both of the wings 101 and 102 of a cushion 100, such as is illustrated in FIG. 12.

The size of a bulge may reduce away from the proximal portion 106c of the connecting wall. For example, an upper part of the nasal bulge 140 of the sectioned cushion 100 of FIG. 12 decreases in size along the upper portion 106a of the connecting wall towards the central portion 103 of the cushion. A nasal bulge 140 may similarly taper in size along the lower portion 106b of the connecting wall towards the central portion 103 of the cushion.

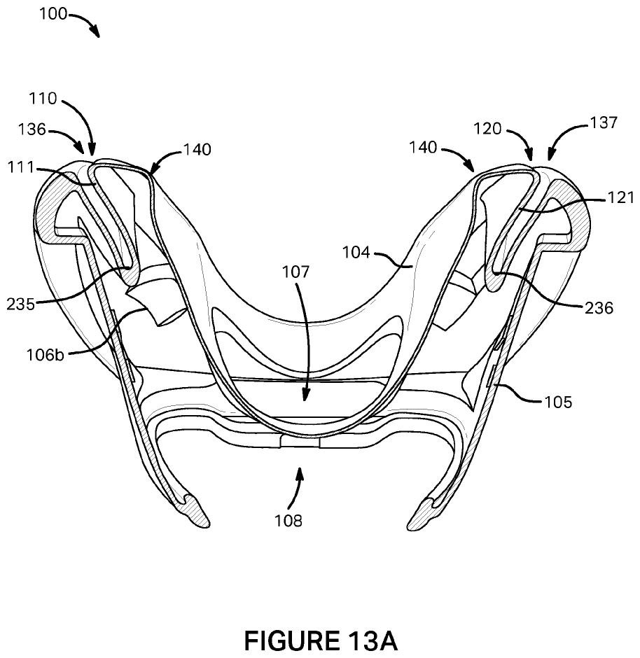

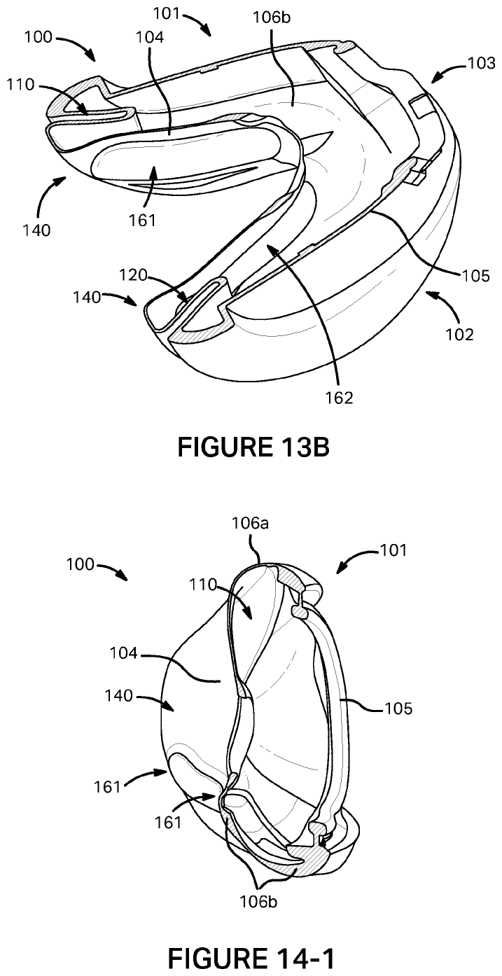

FIG. 13A illustrates a section taken in a downwards direction through a cushion 100. The section is taken along and above the lower portion 106b of the cushion's connecting wall 106. The section is taken through a plane extending through an upper extent of the aperture 108 in the outer cushion wall 105 and just above the nasal aperture 107 of the inner cushion wall 104.

The bases 235 and 236 of the bellows 110 and 120 shown in FIG. 13A are the upper surfaces of the bellows walls joints of the portions of the bellows which extend into the cushion from the respective proximal portions 106c of the connecting wall.

The inner cushion wall 104 may lose sealing engagement with the face of the patient at a region of the inner cushion wall 104 which in use is above the upper lip of the patient. In particular, sealing engagement may be lost along lateral portions of the upper lip region of a patient, either side of the philtrum of the patient's upper lip.

In addition, or alternatively, to the described nasal bulges 140 of the inner cushion wall 104 at or towards the ends of the cushion, various configurations of a cushion 100 may include one or more lip bulges of the inner cushion wall at one or more regions of the inner cushion wall which are to lie along the upper lip region of a patient. A lip bulge is a projection of the inner cushion wall 104 away from the interior of the cushion. By their projecting nature the lip bulges may, when the cushion is worn, generate a locally increased pressure with the patient's face, and thus strengthen the seal of the inner cushion wall with the patient's face. In other configurations, particularly where the inner cushion wall is thinner and/or less stiff at the lip bulges than at adjacent parts of the inner cushion wall, the lip bulges may not cause an increased contact pressure with the patient's face, but instead may operate to collapse and expand as the inner cushion wall 104 moves relative to the patient's face. For example, the lip bulges may be compressed when the inner cushion wall 104 is in normal contact with the patient's face but expand under pressure following a reduction in the contact force from the patient's face on the lip bulges, such as may occur when part of the inner cushion wall 104 is pulled away from the patient's face during side sleeping.

Configurations of a cushion 100 which include one or more lip bulges may include one or more bellows, or may exclude bellows.

Configurations of a cushion 100 which include one or more nasal bulges may include one or more lip bulges, or may exclude lip bulges.

Configurations of a cushion 100 which include one or more bellows may include bulges, such as either or both of nasal bulges and lip bulges.

Lip bulges project outwardly from the body of the cushion at the desired region of the inner cushion wall 104. In some configurations, two lip bulges 161 and 162 may be provided extending along the inner cushion wall 104 proximally from the central portion 103 of the cushion and located beneath a lower extent of the nasal aperture 107.

The inner cushion wall 104 may be thinner at the lip bulges than at least other adjacent parts of the inner cushion wall 104. This may reduce any effect of the lip bulges in causing undesirable pressure concentrations on the patient's face.

Two lip bulges 161 and 162, one associated with each of the respective left wing 101 and right wing 102 of the cushion 100, are illustrated in FIG. 13B. The section view of FIG. 13B shows an opposite part of the cushion 100 from that of the section view of FIG. 13B. The lip bulges 161 and 162 are located on the inner cushion wall 104, proximally extending along the inner cushion wall 104 either side of the central portion 103 of the cushion. The lip bulges are vertically located on the inner cushion wall 104 below the lower lateral extent of the nasal aperture 107, and above a junction of the inner cushion wall 104 with the lower portion 106b of the connecting wall 106.

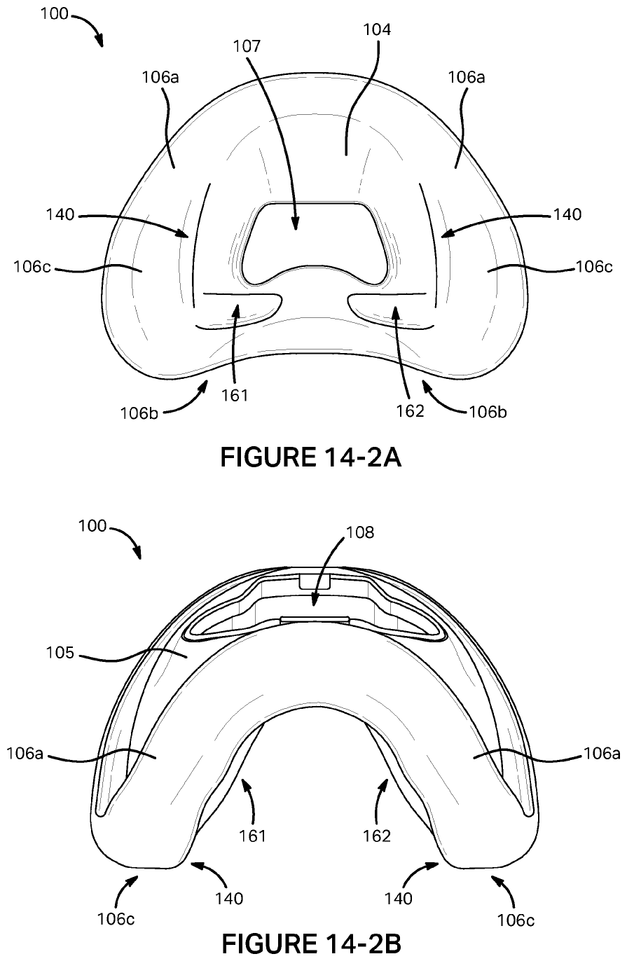

At least parts of the lower portion 106b of the connecting wall 106 may be thickened to support the cushion 100 on the patient's upper lip. The lip bulges 161 and 162 may be located directly adjacent a thickened part of the lower portion 106b of the connecting wall. An example of this is illustrated in FIG. 14-1, which is a section view through the left wing 101 of a cushion 100, at or towards the first end 116 of the bellows 110 and looking towards the end 131 of the wing 101. As seen in FIG. 14-1, a lip bulge 161 is located on the inner cushion wall 104 directly above a thickened part of the lower portion 106b of the connecting wall 106. The left wing 101 of the cushion 100 includes both a nasal bulge 140 and a lip bulge 161, which are formed continuously with each other.

In some configurations, lip bulges 161 and 162 may be oriented to extend along respective sides of the upper lip of the patient and be spaced apart across the central portion 103 of the cushion. The spacing may be such as to receive the philtrum of the patient's upper lip between the distal ends of the lip bulges 161 and 162, at the central portion 103. The absence of a protrusion on the inner cushion wall 104 at the patient's philtrum may reduce the potential for irritation or discomfort for the patient compared to extending the lip bulges across the central portion 103.

In other configurations a lip bulge may extend continuously across the central portion 103 of the cushion, so as to extend across the philtrum of the patient's lip in use.

A cushion according to the disclosure may include one or both of lip and nasal bulges, either in combination with or separately from other cushion features described herein.

For example, FIGS. 14-2A and 14-2B illustrate an embodiment of a cushion 100 that has both nasal bulges 140 and lip bulges 161 and 162. In the embodiment of FIGS. 14-2A and 14-2B the cushion 100 does not include bellows.

As seen in the top view of FIG. 14-2B, the nasal bulges 140 each extend more proximately relative to a patient than do the lip bulges 161 and 162.

While the lip bulges 161 and 162 of the cushion 100 of FIG. 14-2B have a relatively constant width of projection from the inner cushion wall 104 into the space between the two wings of the cushion, in other configurations the width of projection of the lip bulges from the inner cushion wall may vary along the length of the lip bulges.

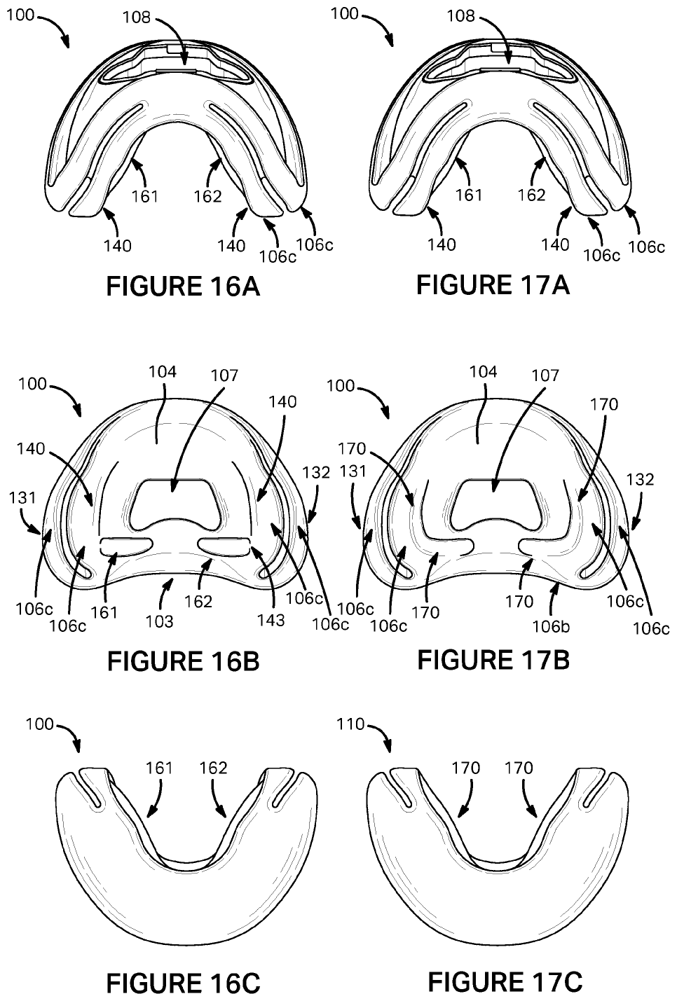

FIGS. 15A-D are section views along the height of both wings of a cushion, at various positions along the extent of each wing. The section of FIG. 15A is taken through each wing 101 and 102 of the cushion 100 proximal to the patient of the extent to which the bellows 110 and 120 extend in depth into the cushion from the proximal portion 106c of the connecting wall 106. The section of FIG. 15B is taken through each wing at a more distal location along each wing 101 and 102 than that of FIG. 16A, but is still proximal of the extent to which the bellows 110 and 120 extend in depth into the cushion from the proximal portion 106c of the connecting wall 106. The section of FIG. 15C is taken approximately mid-way along each of the wings 101 and 102 of the cushion, just proximately of the lateral extent of the nasal aperture 107. Finally, FIG. 15D is taken through the wings 101 and 102 of the cushion just proximally of the aperture 108 of the outer cushion wall 105. In FIG. 15D the whole of the inner cushion wall 104 is shown.

As illustrated in FIGS. 15A-D, each inner cushion wall 104 includes respective lip bulges 161 and 162. The amount the lip bulges 161 and 162 protrude from the cushion increases from their most proximal extent, as seen in FIG. 15A, to a semi-circular projection in cross-section as seen in FIG. 15C. As illustrated in FIG. 15D, the lip bulges 161 and 162 may similarly taper at their most distal ends.

As particularly illustrated in FIG. 15C, the lip bulges 161 and 162 may be located above and extend along a thickened region of the lower portion 106b of the connecting wall. More particularly, the lip bulges 161 and 162 may be located immediately above and connect to a thickened region of the lower portion 106b of the connecting wall.

As also illustrated particularly in FIGS. 15C and 15D, all or part of the lower portion 106b of the connecting wall 106 may be thickened. For example, as illustrated in FIG. 15C the lower portion 106b of the connecting wall is thickened at a part that is directly adjacent to the lip bulges 161 and 162. Such a thickening of part or all of the lower portion 106b of the cushion 100 may provide additional structure to the cushion at the lower portion, and aid in the lower portion supporting the cushion against the patient's upper lip.

Where present together in some configurations of the cushion 100, nasal bulges 140, and lip bulges 161 and 162 may be discrete from each other. For example, as illustrated in FIG. 16B the nasal bulges 140 and lip bulges 161 and 162 of each wing are discrete from each other, with a gap 143 of non-bulged cushion wall between the adjacent portions of the nasal bulges 140 and lip bulges 161 and 162.

In other configurations the nasal bulges 140 and lip bulges 161 and 162 may be, on each respective wing, joined with each other to define a single continuous bulge. An example of this is illustrated in FIGS. 17A-C. The inner cushion wall 104 has combined bulges 170. These continuous bulges 170 continuously extend on the inner cushion wall 104 adjacent to the junction of the inner cushion wall with each of the upper portion 106a, proximal portion 106c, and lower portion 106b of the connecting wall 106.

An inner cushion wall 104 may have the same or similar thickness at a bulge such as nasal bulge 140, as at surrounding regions of the inner cushion wall. For example, as illustrated in FIG. 11, the inner cushion wall 104 at the nasal bulge 140 has a thickness which is approximately the same as the inner cushion wall 104 at a portion directly below the bulge 140, adjacent a lower part of the inner bellows wall 111.

In other configurations the inner cushion wall may be locally thickened at a bulge, to reduce deformation of the inner cushion wall at the bulge and potentially press more firmly against the patient's face at the bulge.

In other configurations the inner cushion wall may be locally thinned at a bulge, to minimise any pressure concentrations against the patient's face. Where the inner cushion wall is locally thinned at a bulge the thinning may be such that the pressurisation of the cushion may cause inflation of the bulge.

In various configurations of a cushion, the inner cushion wall may have one or more thinned regions that do not bulge from the inner cushion wall when the cushion is at rest but are inflated and bulge from the inner cushion wall when the cushion is pressurised. This configuration may be used in addition to or in alternative to any bulges of the inner cushion wall which are present when the cushion is at rest. For example, the inner cushion wall 104 may be thinned at one or more locations along the junction off the inner cushion wall and the upper portion 106a and/or proximal portion 106c of the connecting wall 106 or may be thinned along one or more locations along the junction of the inner cushion wall 104 and the lower portion 106b of the connecting wall 106. These thinned regions may be substantially flush with adjacent parts of the inner cushion wall 104 when the cushion is not pressurised, but when the cushion is pressurised may expand away from the interior of the cushion and, similarly to a nasal bulge 140, allow the inner cushion wall 104 to conform to grooves or folds of the patient's face.

The amount a bulge protrudes from the surrounding cushion wall of a cushion may be varied to provide a desired engagement with a patient's face. For example, the amount a lip bulge protrudes from the surrounding cushion wall may be increased to increase one or both of a pressure the lip bulge exerts against the patient's upper lip in use and an amount of movement of the inner cushion wall 104 relative to the patient's face for which the cushion 104 can maintain a seal with the upper lip of the patient.

The shape and dimensions, including length in a direction along the wings of the cushion, height in a direction between upper and lower parts of the cushion, and amount of medial projection, of a bulge on the cushion wall may also be configured to provide a desired engagement with the patient's face.

For example, lip bulges such as the lip bulges 161 and 162 may be provided having different heights, lengths, and amounts that they project medially. A lip bulge of a relatively lesser height may provide a more vertically localised engagement of the lip bulge with the patient's lip in use than would a lip bulge with a relatively greater height.

FIGS. 19A-C illustrate another embodiment of a cushion 100 having a first bellows 110 and second bellows 120. The cushion 100 has nasal bulges 140 on the inner cushion wall 104 at each wing of the cushion along the upper portion 106a of the connecting wall, and lip bulges 161 and 162 on the inner cushion wall 104 either side of the central portion 103 of the cushion. Compared to the lip bulges 161 and 162 of the embodiment illustrated in FIGS. 17A-C, the lip bulges 161 and 162 of the embodiment of FIG. 19A-C are relatively reduced in the amount to which they medially project. For example, where the lip bulges 161 and 162 of the cushion 100 of FIGS. 17A-C project medially by about 1.5 mm from the adjacent parts of the inner cushion wall, the lip bulges 161 and 162 of the cushion 100 of FIGS. 19A-C may project medially by only about 1 mm.

The nasal bulges 140 and lip bulges 161 and 162 of the cushion 100 of FIGS. 19A-C also each taper from their furthest projecting extents to the adjacent part of the inner cushion wall 104 or connecting wall more gradually than the respective bulges of the cushion 100 of FIGS. 17A-C. While the bulges, and particularly the lip bulges 161 and 162, of the cushion 100 of FIGS. 17A-C may have a substantially semi-circular cross section, the cross sections of the bulges and particularly the lip bulges 161 and 162 of the cushion 100 of FIGS. 19A-C taper away more gradually to the adjacent parts of the inner cushion wall 104 and/or connecting wall 106.

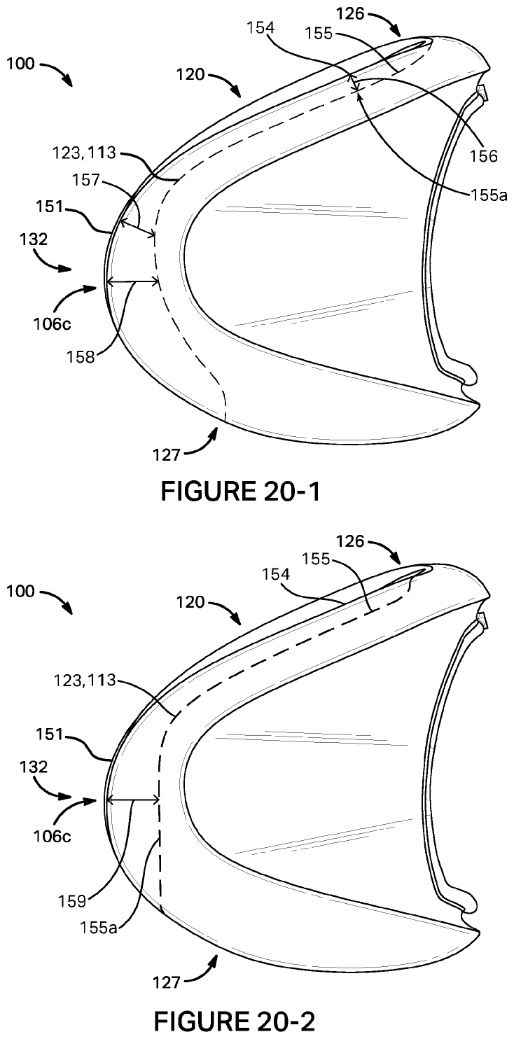

FIG. 20-1 is a side view of a cushion 100. The bellows walls joint 123 of the bellows 120 are illustrated in FIG. 20-1. The bellows 120 has a first end 126 at an upper part of the connecting wall 106.

The depth of a bellows may be measured between the opening of the bellows and the base of the bellows. More particularly, the depth of a bellows may be measured in a direction perpendicular from the connecting wall at the bellows opening to the base of the bellows, being an upper surface of the bellows walls joint 123. For example, in the side view of the cushion 100 of FIG. 20-1, the bellows has a depth indicated at 156 between the point 154 on the upper extremity of the connecting wall and the bellows base 155a.

The depth of the bellows 120 in FIG. 20-1 increases from its first end 126 then follows a consistent depth along a majority of the upper portion 106a of the connecting wall. Towards the end 132 of the right wing, and specifically before reaching the proximal portion 106c of the connecting wall 106, the depth of the bellows 120 increases. The bellows follows this increased depth around the proximal portion 106c and terminates at its second end 127 beneath the proximal portion 106c of the connecting wall on the lower portion 106b of the connecting wall. The bellows towards the second end 127 decreases in depth. This configuration, where the bellows 120 has its greatest depth at and around the end of the wing, may provide for the greatest amount of expansion of the bellows to be located at or around the alar region of the patient.

As illustrated in FIG. 20-1, the bellows 120 has a first depth 156 at a location towards the first end 126 of the bellows. Towards the right end 132 of the cushion the depth of the bellows 120 increases, having a second depth illustrated at 157. At the right end 132 of the cushion, the bellows has a third depth 158. The third depth 158 is greater than both the second depth 157 and the first depth 156.

In some configurations, a cushion 100 with bellows may have the greatest bellows depth at the ends of each wing of the cushion. More particularly, the greatest depth of the bellows of a cushion may be located at respective proximal portions 106c of the connecting wall of the cushion.

As illustrated in FIG. 20-2, the cushion 100 has a bellows 120 in the right wing 102 of the cushion. At the proximal portion 106c of the connecting wall, the bellows 120 has a depth 159 between the connecting wall at the bellows opening 154 and the bellows base 155.

In the configuration of FIG. 20-2, the bellows base at 155a is straight along its length in regions either side of the proximal portion 106c.

For some purposes, the depth of a bellows may also be measured along an axis extending perpendicularly from the bellows base 155 to the connecting wall at the bellows opening 154. When measured in this way, the depth of the bellows 120 in FIG. 20-2 can be said to decrease either side of the depth 159 at the proximal portion 106c of the connecting wall where the bellows walls joint is straight along its length.

While the configuration of a second bellows 120 of the right wing of a cushion has been illustrated and described in relation to FIGS. 20-1 and 20-2, a cushion 100 may include a first bellows 110 on its left wing which is similarly configured to the second bellows 120 of either of FIG. 20-1 or 20-2.

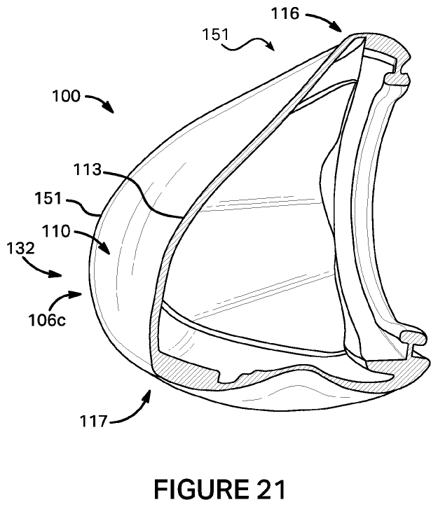

FIG. 21 is a cross-section through the length of a left wing 101 of another cushion 100 which has a first bellows 110. The top 151 of the bellows is shown along with the bellows walls joint 113 of the bellows 110. In the example of FIG. 21 the bellows 110 begin and gradually increase in depth from their first end 116, then continue increasing in depth towards the end 132 of the wing 101, and particularly the proximal portion 106c of the connecting wall. The bellows 110 continues around the end 132 of the bellows and decreases in depth before terminating at the second end 117.

Compared to the bellows walls joint 123 of the bellows of FIG. 20-1, the bellows walls joint 113 along the length of the bellows 110 of FIG. 21 only follow a slight curve between their first end 116 and second end 117.

As illustrated in both FIGS. 20-1, 20-2, and 21, various portions of the bellows have a first radius of curvature of along the length of the bellows walls joints 123 and 113 respectively, and a second radius of curvature along the length of the edge of the cushion at the top 151 of the bellows 120 and 110 respectively.

In some configurations, these first and second radii of curvature of the bellows may be the same, or at least substantially the same, at one or more locations along the length of the bellows.

In other configurations the first radius and second radius of curvature may be different at one or more locations along the length of the bellows.

For example, at least at and adjacent the proximal portion 106c of the connecting wall 106, the second radius of curvature (being of the bellows upper edge at the connecting wall) may be less than the first radius of curvature (being of the bellows base along its length). This relatively lesser radius of curvature of the bellows upper edge about the ends of the cushion means the depth of the bellows is greater there than at respective portions towards each of the first ends 126 and 116 and second ends 127 and 117, respectively, of the bellows.

In particular, in the side view of the cushion 100 of FIG. 21, the bellows walls joints 123 between the first end 126 and second end 127 of the bellows approximates the shape of the top 151 of the bellows, but with a relatively reduced radius of curvature.

While illustrated in FIGS. 20-1, 20-2 and 21 in relation to the second bellows 120 a first bellows 110 of a cushion 100 may be similarly configured.

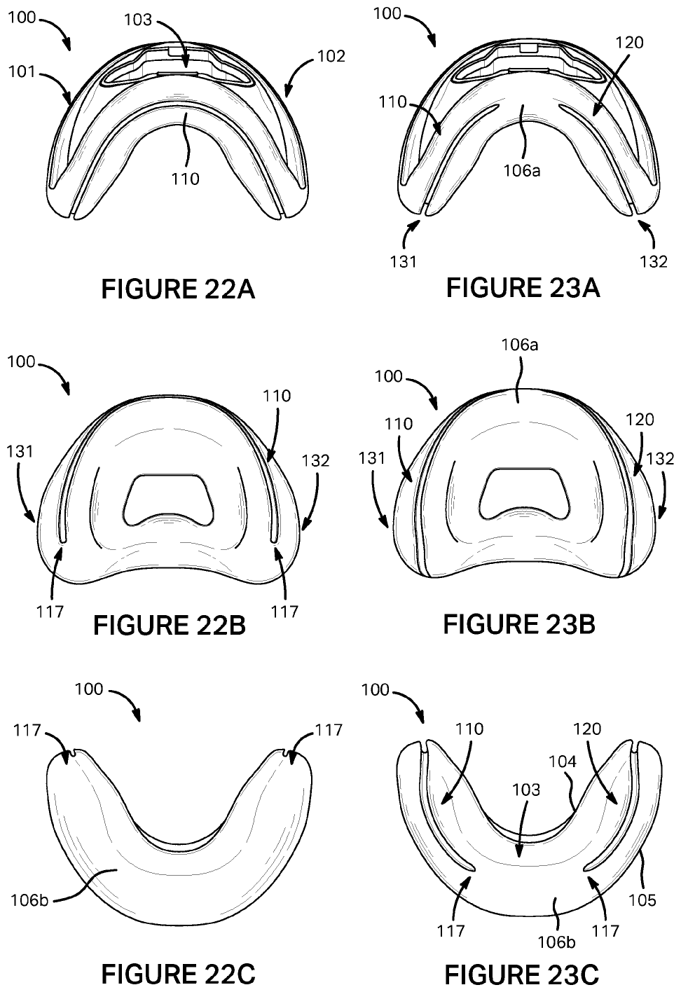

In some configurations the bellows 110 and 120 of respective wings of a cushion may meet across the central portion 103 of the cushion. FIGS. 22A-C illustrate an embodiment of a cushion where a single bellows 110 is provided which in length extends along both wings 101 and 102 of the cushion and across the central portion 103.

The cushion of FIGS. 22A-C has a relatively shallow depth of the bellows 110. A shallow bellows such as this may be desirable where lesser degrees of displacement of the patient interface need to be accounted for.

In the embodiment illustrated in FIGS. 23A-C a cushion 100 with two respective bellows 110 and 120 of either wing of the cushion are of a depth greater than that of the cushion 100 of FIGS. 22A-C but less than that of the cushion 100 of FIG. 16A-C or 17A-C. In particular, the bellows 110 and 120 of the cushion 100 of FIGS. 23A-C extend in depth into the cushion from the ends 131 and 132 of the cushion less than that of the bellows 110 and 120 illustrated in, for example, FIGS. 16A and 17A.

As previously described, according to various configurations of a cushion 100, a bellows may, in addition or alternatively to extending along the upper portion 106a of the connecting wall, extend along the lower portion 106b of the connecting wall 106. FIG. 23A-C illustrates a configuration where the bellows 110 and 120 extend in length along both the upper portion 106a and lower portion 106b of the connecting wall.

In various configurations bellows may be present at part of the upper portion 106a of the connecting wall but not present, or present but not of as much depth as the upper portion bellows, on a corresponding part of the lower portion 106b of the connecting wall. In such configurations when the bellows expand at least some degree of pivoting of the inner cushion wall may result from the unequal depths of the bellows on the upper portion, and, if any, on the lower portion, of the connecting wall. In some configurations this may be acceptable to even desirable, particularly where parts of the patient's face which are prone to losing sealing engagement with the inner cushion wall 104 are at the upper extents of the inner cushion wall.

However, where it is desirable for a lower extent of the inner cushion wall to also move medially when the bellows inflate, the one or both of the bellows may extend in length along at least part or a significant portion of the lower portion 106b of the connecting wall 106 as is illustrated in the embodiment shown in FIGS. 23A-C.

By providing the bellows along a significant part of the lower portion 106b of the connecting wall the way that the inner cushion wall 104 moves when the bellows expand may be changed. In particular, as the depth of any bellows on the lower portion of the connecting wall increases, any pivoting of the inner cushion wall 104 about the lower portion 106b of the connecting wall may be reduced. Where the bellows on the upper portion and lower portion of the connecting wall are of equal depth at vertically adjacent parts of the inner cushion wall 104 and outer cushion wall 105 the expansion of the bellows may move the inner cushion wall 104 and outer cushion wall 105 relative to each other without any vertical pivoting.

A joint between the bellows walls of a cushion, and/or between a respective bellows wall of a cushion and an adjacent part of the connecting wall 106, may have or a portion of it may have, a radius of curvature between the parts the joint connects.

The size of the radius of curvature of a joint may impart function to the joint. For example, a joint with a relatively increased radius of curvature may allow for relatively increased total expansion and contraction of a bellows associated with the joint. An increased amount of expansion and contraction of a bellows may increase the ability of the cushion to maintain an effective seal with the patient's upper lip when the cushion is internally pressurised by a therapy airflow.

The size of the radius of curvature of a joint may also impact the comfort of the cushion for a patient. For example, where a bellows walls joint contacts the patient's face in use, a relatively increased radius of curvature of that joint may provide a relatively more gradual pressure gradient on the patient's face from the inner cushion wall, across the joint, and towards the opening of the bellows than would a joint with a smaller radius of curvature.

The radius of curvature of a bellows walls joint at the base of a bellows, which does not contact the patient's face in use, may also impact the comfort of the cushion for a patient. A relatively larger radius of curvature at a bellows base may cause the bellows opening to be wider in use than with a bellows base of a relatively smaller radius of curvature.

Where one or more of the joints of a bellows has a radius of curvature, that radius may be the same along the length of the bellows.

In other configurations, the radius of a curvature of a joint of a bellows may vary along the length of the bellows.

For example, along a bellows at an upper portion 106a of the connecting wall 106 of a cushion, at least the bellows walls joint 113 may have a relatively smaller radius of curvature than the bellows walls joint of a bellows at a corresponding lower portion 106b of the connecting wall. In some configurations, each of the bellows walls joint 113 and upper joints 114 and 115 of a bellows on an upper portion 106a of the connecting wall of a cushion may have a larger radius of curvature than that of the corresponding bellows walls joint 113 and upper joints 114 and 115 of a bellows on a lower portion 106b of the connecting wall of the cushion.

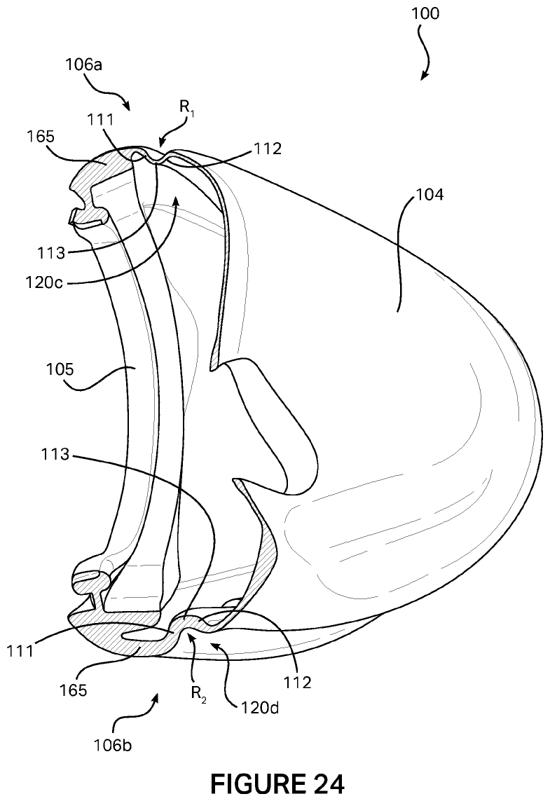

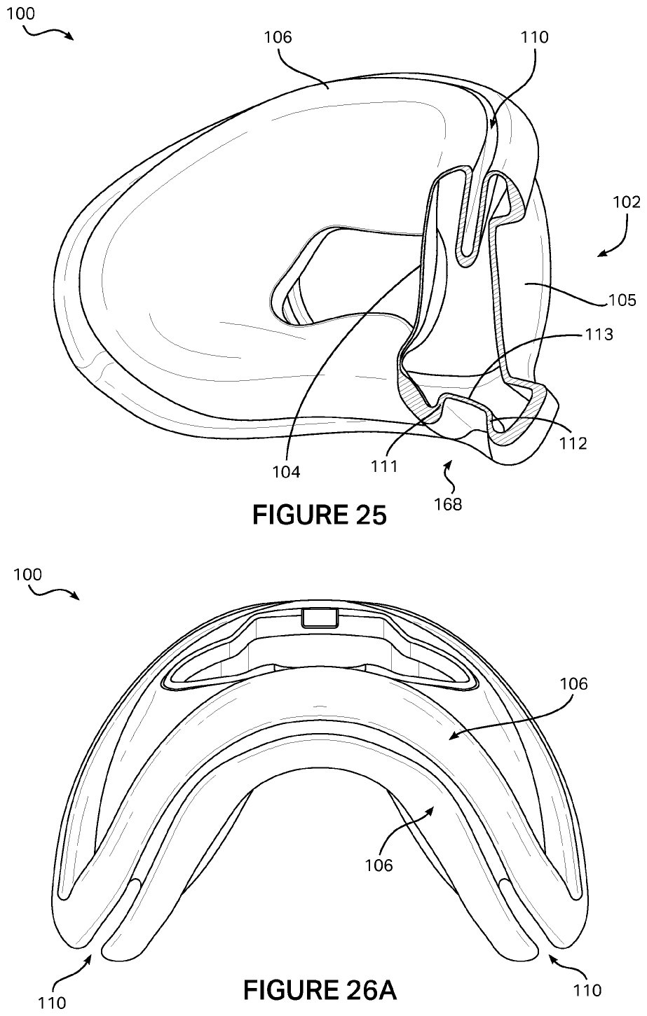

FIG. 24 is a cross-sectional view taken through the height of the right wing 102 of a cushion 100. The cushion 100 has a bellows 120 which is present along both the upper portion 106a and lower portion 106b of the connecting wall 106. At the section of FIG. 24, the bellows walls joint 113 at an upper portion 120c of the bellows at the upper side of the cushion has a first radius of curvature R₁. At the corresponding lower part of the section of FIG. 24, the lower portion 120d of the bellows walls joint 113 at the lower side of the cushion has a second radius of curvature R₂.

In FIG. 24 the first radius of curvature R₁ at the upper portion 106a of the connecting wall is smaller than the second radius of curvature R₂ at the lower portion 106b of the connecting wall. The relatively smaller first radius of curvature R₁ may reduce the visual prominence of the bellows at the upper side of the cushion.

Where a radius of curvature of a bellows walls joint at a lower portion of a cushion is larger than the radius of curvature of a corresponding bellows walls joint at an upper portion of the cushion, it may be larger by, for example, about 5% to about 25%.

For example, where a bellows walls joint at an upper portion of the cushion has a radius of curvature of about 1.4 mm, a corresponding bellows walls joint at a lower portion of the cushion may have a radius of curvature of about 1.6 mm.

When a flexible cushion is retained on a patient's face as part of a patient interface, the stability of the cushion may predominantly be influenced by the stability of the lower portions of the cushion. This may particularly be accentuated by tube drag or other forces exerted on the cushion during use.

To provide a cushion that is flexible, but which retains its stability in use, lower portions of the cushion may have a relatively greater thickness than upper portions of the cushion.

For example, a lower portion 106b of a connecting wall of a cushion may have a greater thickness than an upper portion 106a of the connecting wall of the cushion.

Where a cushion includes bellows at both the upper portion 106a and lower portion 106b of the connecting wall 106, one or more parts of the bellows at the lower portion may be thicker than one or more parts of the bellows at the upper portion.

Thickening the bellows at the lower portion of the connecting wall may ensure that the bellows better resist vertical movement of the inner cushion wall 104 and outer cushion wall 105 relative to each other.

More particularly, the bellows walls and bellows walls joint of a bellows at a lower portion 106b of the connecting wall may each have a greater thickness than a respective one or each of the bellows' walls and bellows walls joint of a bellows at an upper portion 106a of the connecting wall.

As illustrated in FIG. 24, the lower portion 120b of the bellows 120 has relatively thicker bellows walls 111 and 112 and bellows walls joint 113 than does the upper portion 120a of the bellows 120.

The upper joints of the bellows walls with the connecting wall may also be relatively thicker at a lower bellows than at an upper bellows.

Where the cushion includes a locally thickened section to provide support to the cushion, the thickness adjacent parts of a bellows may be able to be relatively reduced.

For example, as seen in FIG. 24 the connecting wall 106 of the cushion 100 includes a thickened portion 165 adjacent to the outer cushion wall 105. The stability afforded by the thickened portion 165 may allow the walls and joints of the bellows 120 at both the upper portion 106a and lower portion 106b of the connecting wall to be relatively reduced, while still maintaining the same overall stability of the cushion 100.

Reducing the thickness of one or more parts of a bellows provides increased flexibility of the bellows. This may allow the bellows to expand under a reduced internal pressure of the cushion. When the cushion is used as part of an interface on a patient's face, a bellows of reduced thickness may more readily expand against the patient's face when a contact force from the patient's face reduces.

While reducing the thickness of the whole cushion, or whole regions of the cushion, may compromise the structural integrity of the cushion, providing relatively thinned parts of the cushion adjacent to relatively thickened parts may provide the benefits of increased bellows flexibility without undesirably destabilising the cushion.

A cushion with a bellows as described herein may provide improved contact of the inner wall with the patient's face in use, particularly during side-sleeping. While retaining the inner cushion wall to the patient's face at the non-laid-on side of the cushion may help improve comfort and compliance, the pressure of the cushion against the patient's nose on the laid-on side of the cushion may constrict or even close the patient's nostril on that side. This may cause patient discomfort, and/or reduce the efficacy of the therapy.

A constricting or closing force on a nostril during side-sleeping may particularly be caused by the collapse of the cushion adjacent to the nostril, so that the outer cushion wall is pressed against the associated side of the patient's nose.

A cushion according to the disclosure may include bellows which has variable spacing between its bellows walls. In particular, a bellows of a cushion may have one or more widened regions in which the spacing of the bellows walls from each other is locally increased. This increased spacing of the bellows walls may help prevent the outer cushion wall 105 acting on the inner bellows wall 104 in use, such as during side sleeping, which may otherwise act on the patient's nose and constrict one of their airways.

As previously described, a bellows walls joint may have a curved shape, and where it is a continuously curved shape may define one or more radii of curvature between the bellows walls which it connects.

In other forms, a bellows walls joint may take other shapes, such as a flat section.

FIG. 25 is a view of a cushion 100 with a section taken through the height of the right wing 102 of the cushion. In the illustrated embodiment, the cushion 100 has a single continuous bellows 110 which extends about the connecting wall 106 between the inner cushion wall 104 and outer cushion wall 105. At the lower portion 106b of the connecting wall at the location of the section of FIG. 25, the bellows 110 has a locally widened spacing between the bellows walls 111 and 112. This defines a widened portion 168 of the bellows. At the widened portion 168, the bellows walls joint 113 is of locally increased width. The bellows walls joint 113 at the widened portion 168 provides the bellows with a flat bellows base between the two bellows walls 111 and 112.

When experiencing a force compressing the inner cushion wall 104 and outer cushion wall 105 towards each other at the right wing 102 of the cushion, the bellows walls 111 and 112 may be caused to pivot towards each other about the bellows walls joint 113, further contracting the lower portion of the bellows 110 at the widened portion 168 from the at rest condition illustrated in FIG. 25.

In addition, or alternatively, to allowing an increased contraction of the bellows by greater pivoting of the bellows walls towards each other, the widened bellows walls joint 113 may itself flex or deform under forces pushing the inner cushion wall 104 and outer cushion wall 105 towards each other. Any such deformation of the bellows walls joint may increase the total amount of contraction that the bellows can provide from its resting condition.

Contraction of the bellows may act to absorb energy from the compressing force on the wing of the cushion, so that a pressure experienced by the inner cushion wall 104 on the patient's nose is reduced. This may reduce the potential for pressure from the inner cushion wall 104 to restrict one of the patient's nasal airways.

The bellows walls joint at a widened portion 168 may act to sacrificially collapse and so absorb energy under compression.

In other forms, the bellows walls joint at a widened portion 168 may be configured to not deform, or to have limited deformation, under a forcing of the inner cushion wall 104 and outer cushion wall 105 towards each other.

A widened portion 168 to provide increased spacing between the walls of a bellows may be provided at any desired location on the cushion, and particularly at a location where use forces may urge the structure of the cushion to collapse.

A widened portion 168 of a bellows may be provided along regions of the bellows which are adjacent to the ala nasi of the patient's nose in use. In this location, the local widenings may act to reduce pressure on the side of the patient's nose that would constrict one of the patient's nasal airways.

A widened portion 168 of a bellows may be provided on each wing of the cushion on the bellows at the lower connecting wall such that the local widening proximally and distally spans the point to which the bellows extend into the cushion from the proximal ends of the cushion.

A widened portion 168 of a bellows may be provided on each wing of the cushion on the bellows at the lower connecting wall, distally of the extent to which the bellows extend into the cushion from the proximal ends of the cushion.

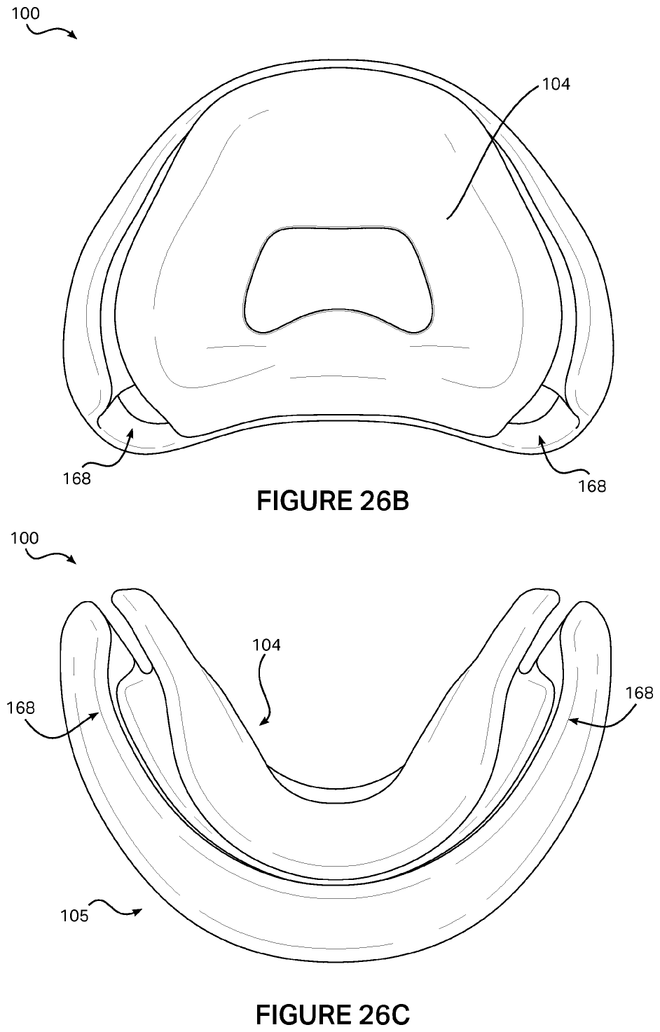

FIGS. 26A-C are, respectively, a top view, rear view, and bottom view of the cushion 100 of FIG. 25, having respective widened portions 168 of the bellows.

As seen in FIG. 26A, the cushion 100 has a bellows 110 that extend along the connecting wall 106 at both the left and right wings of the cushion.

As illustrated in FIG. 26C, the width of the bellows gradually increases then decreases across the length of the widened portion 168. As illustrated in FIG. 26C, the widened portion 168 begins towards the ends of the cushion and terminates part way along each wing of the cushion towards the cushion's middle.

More particularly, as illustrated in FIG. 26C the widening of the bellows on the lower portion 106b of the connecting wall 106 begins further towards the lateral ends of the cushion than where the bellows extend into the cushion from the lateral ends of the cushion.

For a bellows to expand and optionally contract as desired, the walls and/or joints of a bellows may be relatively flexible. However, such flexibility may reduce the overall stability of the cushion, and particularly the stability of the inner cushion wall 104 and outer cushion wall 105 relative to each other. For example, particularly where a bellows extends around a substantial portion or all of the connecting wall, the flexibility of the bellows may reduce the resistance of the inner cushion wall 104 and outer cushion wall 105 to translate relative to each other. In use, such reduced translative stability may result in reduced stability on the patient's face of an interface that includes the cushion.

A cushion may include one or more reinforcing ribs that span at least part of the connecting wall of the cushion. For example, one or more reinforcing ribs may be provided as part of or on the lower portion 106b of the connecting wall 106 of the cushion. At the reinforcing ribs the connecting wall is provided with a locally increased resistance to the movement of the inner cushion wall and outer cushion wall towards each other.

Where a cushion includes a bellows, a reinforcing rib, particularly where it is provided across the bellows, may provide increased stability to the cushion by inhibiting translation of the inner cushion wall 104 and outer cushion wall 105 relative to each other.

A cushion may include two reinforcing ribs, each located on the connecting wall of the cushion to be, in use, adjacent to and below the ala nasi of each side of the patient's nose. Accordingly, the reinforcing ribs of a cushion may be located one on each wing of the cushion.

The reinforcing ribs of a cushion may be located on the connecting wall of a cushion below the respective lateral extents of the nasal aperture 107 in the inner cushion wall 104 of a cushion.

The reinforcing ribs of a cushion may be located laterally away from the respective lateral extents of the nasal aperture 108 of the inner cushion wall 104 of a cushion.

Reinforcing ribs provide increased stiffness to the cushion.

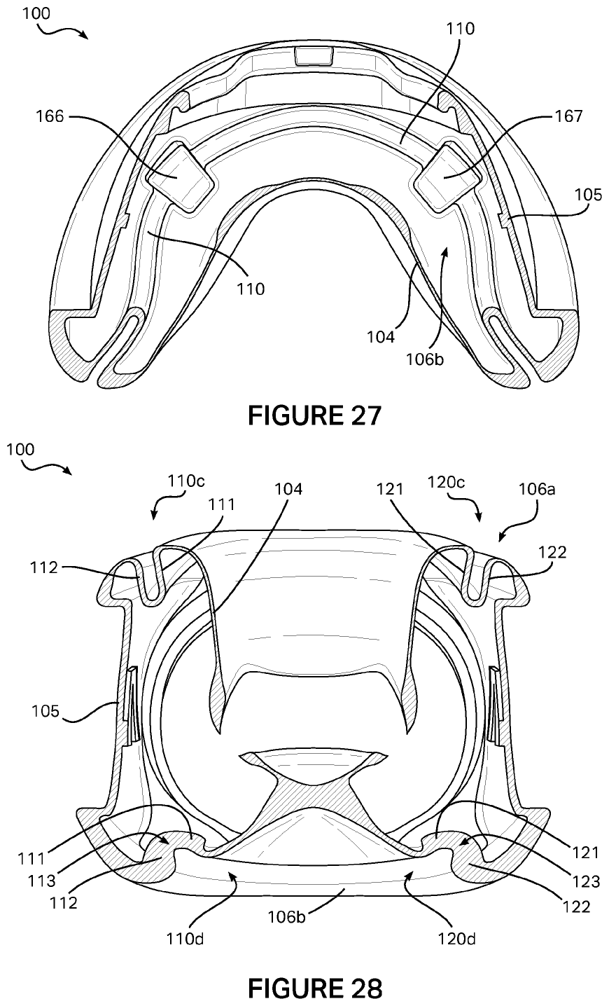

FIG. 27 is a sectional view through the height of a cushion 100, showing the lower half of the cushion. The cushion 100 includes a bellows 110 on the lower portion 106b of the connecting wall 106 along both lateral wings of the cushion. The cushion 100 includes two reinforcing ribs 166 and 167. The reinforcing ribs 166 and 167 each span across the width of the bellows 110.

Reinforcing ribs may define a locally thickened portion of the cushion, and more particularly of the connecting wall 106. As illustrated in FIG. 27, the reinforcing ribs 166 and 167 thickened portions provided to the connecting wall 106. In other configurations, reinforcing ribs may be provided as separate elements which are attached to the internal surface of the connecting wall 106 in the desired location or locations.

A reinforcing rib which is formed as part of the connecting wall may have a thickness that is, for example, about 2 to about 5 times the thickness of one or more reinforcing-rib-adjacent parts of the connecting wall. For example, the reinforcing rib may have a thickness at the bellows that is about 2 to about 5 times the thickness of the bellows walls and/or bellows walls joint.

In at least some forms the thickness of the rib extends into the interior of the cushion. The exterior surface of the cushion, for example the external surface of the connecting wall 106 in the configuration of FIG. 27, may be unchanged by the presence of one or more reinforcing ribs.

The width of a reinforcing rib in a direction along the length of the bellows may correspond to a proximal-distal dimension of an ala nasi of a patient. For example, the reinforcing rib may have a width of about 5 mm to about 10 mm.

In some configurations the width of a reinforcing rib may be less than the proximal-distal dimension of an ala nasi of a patient.

While one or more reinforcing ribs may be utilised in a cushion to impart increased stability to the cushion, reinforcing ribs may additionally or alternatively be utilised to resist lateral pressure on the interface causing constricting or closing off an airway of the patient.

The walls of the bellows of a cushion may be configured in different conditions relative to each other when the cushion is at rest. As previously described, the walls of a bellows may be substantially plane-parallel with each other when at rest. In other forms, the bellows walls may be configured to converge towards each other away from their bellows walls joint, so that in an at rest condition the opening to the bellows is narrowed comparative to a width at the base of the bellows. In particular, in some forms in an at rest condition the opening of the bellows may be closed.

In other forms, the walls of a bellows may diverge from each other away from the bellows base when the cushion is in an at rest condition.

Bellows walls which diverge from each other will cause the opening of the bellows to be widened compared to a bellows with plane-parallel walls.

Bellows walls which diverge from each other may also simplify the manufacture of the cushion as a single moulded part, enhancing mould release from the moulded cushion.

Where the depth of a bellows varies along its length, the diverged distance between the base of the bellows and bellows opening may be constant along the length of the bellows, while the angle of the bellows walls relative to each other varies. In other configurations of a bellows with a depth that varies along its length, the angle of the bellows walls to each other may be constant while the distance between the bellows walls at the bellows base and bellows opening may vary.

In other configurations both an angle between the bellows walls and the difference in spacing of the bellows walls between the bellows base and bellows opening may be variable along the length of a bellows.

The walls of a bellows may diverge from each other away from the base of the bellows only at some locations on the cushion.

FIG. 28 is a sectional view through a cushion 100, showing both an upper portion 110c of a first bellows 110, and upper portion 120c of a second bellows 120. Also shown are in the section of the cushion are respective lower portions 110d and 120d of each of the bellows. The lower portion 110b of the first bellows 110 and lower portion 120b of the second bellows 120 are connected together to form a continuous bellows along the lower portion 106b of the connecting wall 106.

The cushion 100 of FIG. 28 is shown in an at rest condition.

At the lower portion 106b of the connecting wall the bellows walls 111 and 112 are non-parallel with each other. The bellows walls 111 and 112, and 121 and 122, diverge away from each other from their ends at the respective bellows walls joints 113 and 123 to the opening of the bellows.

In the configuration illustrated in FIG. 28, the external surfaces of the bellows walls 111 and 112, and 121 and 122, at the lower portion 106b of the connecting wall are angled about 90 degrees from each other about the respective bellows walls joints 113 and 123.

In the configuration of FIG. 28 the external surfaces of the bellows walls 111 and 112, and 121 and 122, at the upper portion 106a of the connecting wall are substantially parallel with each other when the cushion is in an at rest condition.

In other configurations however, the bellows walls 111 and 112, and 121 and 122, at an upper portion 106a of the connecting wall of a cushion may also diverge from each other away from their common bellows walls joint.

As previously described, particularly in relation to the cushion 100 of FIG. 9A-B, the opening of a bellows at the ends of each wing of the cushion and/or portions of the length of the bellows towards the ends of each wing may be oriented laterally outwards. As described, this may define a kink in the bellows between a first region and second region, particularly when viewed from above or below the cushion.

Further examples of configurations of cushions including kinks between proximal and lateral regions of the bellows along each wing of the cushion are shown in FIGS. 16A-C, 17A-C, 23A-C, 25-26C, 27, and 29A-C.

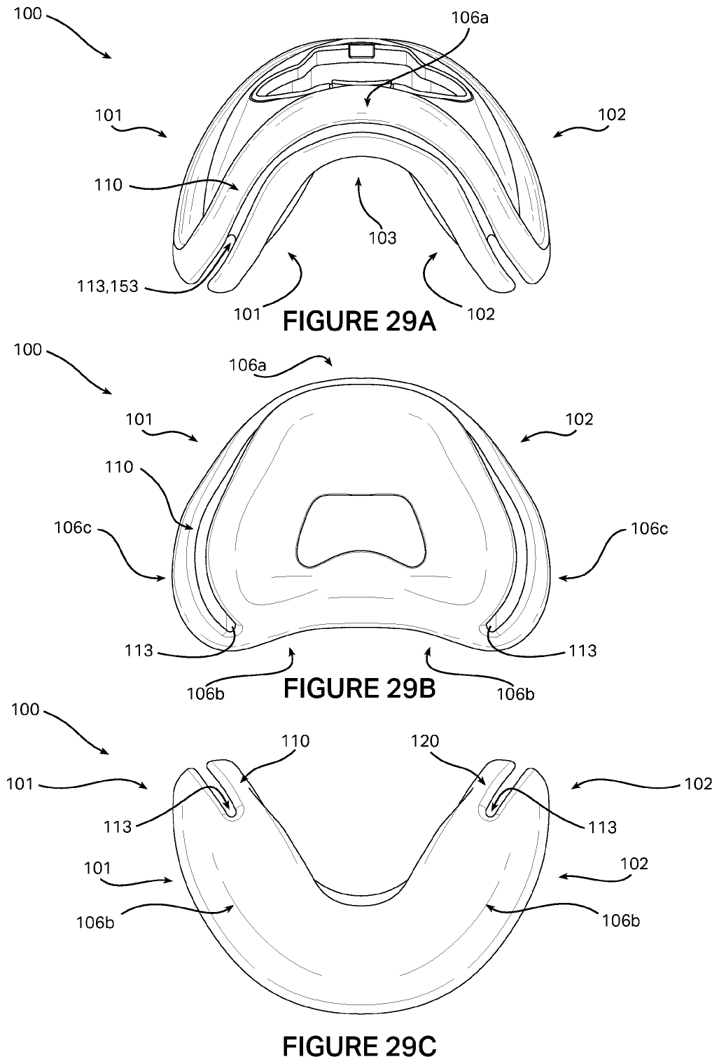

FIG. 29A is a top view, FIG. 29B a rear view, and FIG. 29C a bottom view of another configuration of a cushion 100. The cushion 100 has a bellows 110 which extends along the connecting wall 106 at both the left wing 101 and right wing 102 of the cushion.

The bellows 110 extends around both the respective left and right ends of the cushion, but does not extend along the lower portion 106b of the connecting wall.

Where a or the bellows of a cushion is not present at the lower portion 106b of the connecting wall, the base 153 of the bellows does not extend in the same direction as the surface of the lower portion 106c of the connecting wall. The base 153 of the bellows is the upper surface within the bellows of the bellows walls joint 113.

In the configuration of FIGS. 29A-C, and particularly as seen in FIGS. 29B and 29C, the bellows walls joint 113 does not extend along the lower portion 106c of the connecting wall. Instead, the bellows walls joint 113 terminates at the lower connecting wall.

More particularly, at the lower connecting wall at each wing of the cushion, the adjacent bellows walls joint 113 is oriented substantially perpendicularly to the surface of the lower connecting wall 106.

Where a bellows extends along part of the connecting wall 106, its expansion causes the parts of the connecting wall 106 either side of the bellows to translate away from each other. For example, in the configuration of FIGS. 29A-C, an expansion of the bellows 110 causes the parts of the upper portion 106a and proximal portion 106c of the connecting wall to translate away from each other. As the bellows 110 terminates at the lower portion 106b and does not extend along them, an expansion of the bellows 110 does not cause respective parts of the lower portion 106b of the connecting wall to translate away from each other. Instead, the lateral portions of the lower portion 106b are caused to pivot relative to each other when the bellows 110 expands.

Without a bellows which extends along the lower portion 106c of the connecting wall the stability of a cushion may be increased.

At each of the left and right ends of the cushion the bellows 110 extend into the cushion. As seen particularly in FIGS. 29A and 29C, at the ends of the cushion the bellows define an opening between the upper portion 106a and lower portion 106b of the connecting wall 106.

The extension of the bellows into a cushion from its ends is illustrated, for example, by the section views of FIGS. 15A and 15B.

As seen in FIG. 29A, bellows 110 at the ends of the cushion extends into the cushion so that from the proximal portion 106c of the connecting wall the base 153 of the bellows is located about ¼ to about ⅓ from the end of each wing of the cushion.

The depth of the bellows along the upper portion 106a of the connecting wall may be less than the bellows depth at the ends of the cushion.

In particular, a depth of the bellows along the upper portion 106a of the connecting wall 106 may be about ⅕th to about ¾ of the depth of the bellows at the ends of the cushion.

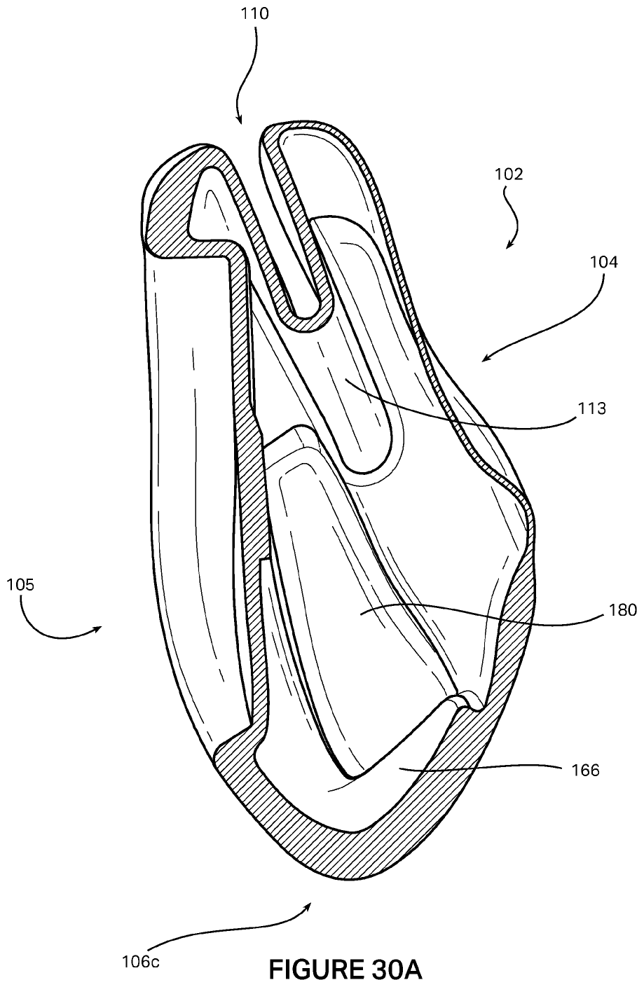

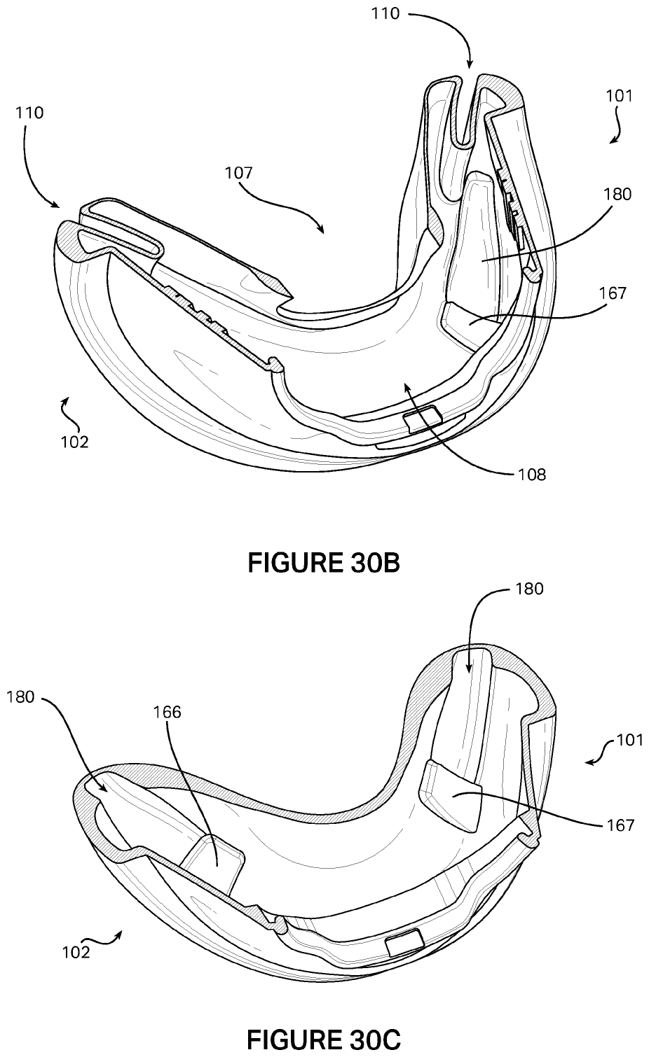

Various section views of a cushion 100 are illustrated in FIGS. 30A-C. The cushion 100 of FIGS. 30A-C may for example be the cushion 100 illustrated and described in relation to in FIGS. 29A-C.

In FIG. 30A the section illustrated is taken transversely through the height of the right wing 102 of the cushion and shows the view at the section towards the end of the right wing 102.

As seen in FIG. 30A, the cushion 100 has a bellows 110 for providing expansion of the cushion at the end of the right wing. The bellows walls joint 113 extends substantially perpendicularly upwards from the lower portion 106c of the connecting wall.

At the section of FIG. 30A, the lower portion 106c of the connecting wall includes a reinforcing rib 166. The reinforcing rib 166 extends across the lower portion 106b of the connecting wall and to the inner cushion wall 104 and outer cushion wall 105. The cushion 100 of FIG. 30A does not include a bellows extending along its lower portion 106b of the connecting wall.

Also visible in FIG. 30A is a thinned region 180 of the cushion. The thinned region 180 is located along the lower portion 106c of the connecting wall.

A thinned region may provide relatively increased flexibility to the cushion.

The thinned region comprises a groove in the cushion. The groove may extend into the wall of the cushion from either the inside of the cushion or from the outside of the cushion.

A thinned region may have a generally rectangular shape, with a length oriented along the length of the wing of the cushion. As seen in FIG. 30A, the groove of the thinned region extends along the length of the cushion from the reinforcing rib 166. The width of the groove decreases away from the reinforcing rib 166 towards the end of the wing of the cushion. The end of the groove away from the reinforcing rib 166 extends beyond the bellows walls joint 113 where it extends upwardly from the lower portion 106c of the connecting wall 106. As seen in FIG. 30A, the end of the groove away from the reinforcing rib 166 is located on the outer cushion wall 105 side of the bellows wall joint 113.

At its end adjacent the reinforcing rib 166, the width of the thinned region 180 between the inner cushion wall 104 and outer cushion wall 105 is less than that of reinforcing rib 166.

In some configurations the thickness of the cushion wall at a thinned region may be consistent across the thinned region.

In other configurations the thickness of the cushion wall at a thinned region may vary across the thinned region.

The thinned region allows decoupled movement of relatively thicker cushion wall portions on either side of the groove.

A thinned region may be provided in combination with one or more reinforcing ribs.

The section of FIG. 30B is taken across the height of the cushion, at approximately the middle of the cushion 100. The inside of the cushion and the inside surface of the lower portion 106c of the connecting wall are shown.

The left wing 101 of the cushion 100 is illustrated in FIG. 30B as including a bellows 110 configured to allow expansion of the end of the wing, and a reinforcing rib 16 as described in relation to the section of the right wing 102 of the cushion in FIG. 30A.

As seen in FIG. 30B, the reinforcing rib 167 is located beneath and extend laterally of the lateral extents of the nasal aperture 107 of the inner cushion wall 104. The thinned region 180 extends from reinforcing rib 167 along the left wing 101 of the cushion towards its end.

The width of the reinforcing rib 167 between the inner and outer cushion walls is greater than the width of the thinned region 180.

In various forms, reinforcing ribs may be located either medially of, beneath, or laterally of the lateral extents of the nasal aperture 107.

To improve comfort, it may be desirable to locally reduce the thickness of the cushion in one or more regions adjacent the patient's nose and upper lip. For example, the inner cushion wall and lower portion of the connecting wall may be relatively thinned or may include thinned regions. A relatively thinner cushion wall will be more supple and may more readily conform to the patient's face. However, reducing the thickness of the cushion wall will reduce its structural integrity.

One or more reinforcing ribs may be utilised to provide locally increased structure to the cushion. The reinforcing ribs may particularly be located within or adjacent to part of the cushion wall which is of a relatively reduced thickness.

As seen in FIG. 30B, the reinforcing rib 167 is located beneath the lateral extent of the nasal aperture, so as to increase the structural integrity of the cushion at a corresponding upper lip region of the patient in use.

The section of FIG. 30C is taken across a lower height of the cushion than the section of FIG. 30B. The section of FIG. 30C is taken through part of the thinned region 180 of each wing of the cushion.

While generally described as having different wall sections, a cushion and the bellows of that cushion may be formed as a single moulded item. Parts of the cushion may blend with each other or merge into each other. For example, an inner cushion wall may blend into the connecting wall of the cushion, rather than provide a distinct junction or transition. Or, as another example, the walls of a bellows may at least at some parts lack a defined planar-form wall, and instead be part of a region of the cushion which curves from the bellows base to the upper edge of the bellows.

Accordingly, the parts of a cushion should generally, unless the context dictates otherwise, be understood in reference to their described function. For example, the walls of a bellows may be understood to be the portions of the cushion which open away from each other about a common bellows walls joint when the bellows expand. Or, as a further example, the inner cushion wall may be understood to be a part of the cushion which is to contact the patient's face in use, while the outer cushion wall is to face away from the patient's face and in some configurations be supported by a frame. Within this context, the connecting wall would be generally understood to be a part of the cushion which connects together the inner cushion wall and outer cushion wall.

While generally described and illustrated as having a single fold, with one set of bellows walls which are jointed to each other, the bellows of a cushion may include multiple folds. In such a configuration there would be three, four, or more, bellows walls, each of which is jointed to an adjacent one and the most inner and outer bellows walls are jointed to the connecting wall. In a configuration with multiple folded bellows the bellows walls would open like an accordion.

Utilising multiple folds may allow for a desired amount of expansion to be provided by the bellows while decreasing a necessary depth of the bellows compared to a cushion having a single-folded bellows.

Utilising multiple folds may also allow for an increase in the amount of expansion which a bellows can offer compared to a single-fold bellows.

While generally described as individual bellows associated with each wing of the cushion, such bellows of a cushion may be separate from each other or may be continuous with each other. For example, illustrated in for example FIG. 22A-C, or 29A-C, a cushion may have a continuous bellows which extends along at least the upper connecting wall, and potentially also about the ends of the cushion and to or along the lower connecting wall. In other configurations, for example as illustrated in FIG. 23A-C, the bellows of a cushion may not be continuous along the upper connecting wall, and the cushion may define two or more non-continuous bellows. Or, in still other configurations, a cushion may include multiple bellows associated with each wing of the cushion. An example of this is illustrated in FIGS. 18A-C, where the cushion 100 has bellows 110 and 210 associated with the left wing 101 of the cushion, and bellows 120 and 220 associated with the right wing of the cushion.

As illustrated in FIGS. 18A-C, each of the bellows 110 and 210, and 120 and 220, are distinct and non-continuous from each other. However, in other configurations, some or all of the bellows of the cushion 100 of FIGS. 18A-C may extend continuously into each other.

A cushion according to the disclosure may be formed as a single moulded part. For example, the cushion may be injection moulded from a plastics or silicone material. Accordingly, all the parts of the cushion, including the inner cushion wall 104, outer cushion wall 105, and connecting wall 106 may be unitarily formed with each other. Furthermore, the bellows of a cushion, for example inner bellows wall 111, outer bellows wall 112, bellows walls joint 113, and upper joints 114 and 115 of a first bellows 110, and inner bellows wall 121, outer bellows wall 122, bellows walls joint 123, and upper joints 124 and 125 of a second bellows 120 may be unitarily formed with each other and the other walls of the cushion.

The inner cushion wall may have a lesser thickness than the outer cushion wall. For example, the outer cushion wall may have a thickness of about 0.8 mm while the inner cushion wall may have a thickness of about 0.3 mm.

The lower portion 106b of the connecting wall may have a greater thickness than the inner cushion wall 104. For example, the inner cushion wall may have a thickness of about 0.3 mm while below the lowest extent of the inner cushion wall the lower portion 106b of the connecting wall may have a thickness of greater than 0.3 mm.

Where a cushion 100 includes lip bulges 161 and 162 which are located on the inner cushion wall 104 adjacent to the lower portion 106b of the connecting wall, the lower sides of the lip bulges 161 and 162 may connect to the any thickened region of the lower portion 106b of the connecting wall. An example of this is illustrated in FIG. 15C, where the lower sides of the lip bulges 161 and 162 each depend from a thickened region of the lower portion 106b of the connecting wall.

As described and illustrated in relation to various embodiments the bellows of a cushion may be spaced apart across the central portion of the cushion.

According to some embodiments the first ends 116 and 126 of the first and second bellows may be spaced apart across the central portion 103 by, for example, about 1 mm to about 30 mm.

Although embodiments have been described with reference to a number of illustrative embodiments thereof, it will be understood by those skilled in the art that various changes in form and details may be made therein without departing from the spirit and scope of the invention as defined by the appended claims. Therefore, the preferred embodiments should be considered in a descriptive sense only and not for purposes of limitation, and also the technical scope of the invention is not limited to the embodiments. Furthermore, the present invention is defined not by the detailed description of the invention but by the appended claims, and all differences within the scope will be construed as being comprised in the present disclosure.

Many modifications will be apparent to those skilled in the art without departing from the scope of the present invention as herein described with reference to the accompanying drawings.

1. A nasal cushion for a respiratory interface, the cushion having a central portion and left and right wings that extend to respective left and right lateral ends from the central portion, the cushion comprising:
an inner cushion wall to contact a patient's face,
a non-face contacting outer cushion wall,
a connecting wall that connects the inner cushion wall and outer cushion wall, and
a bellows extending in length along the connecting wall and in depth into the cushion at each of the left and right wings of the cushion,
wherein the or each of the bellows are expandable to increase a spacing between the inner cushion wall and outer cushion wall.

2. The nasal cushion of claim 1, wherein the or each of the bellows respectively extend in length from each of the left and right lateral ends of the cushion along the left and right wings towards the central portion.

3. The nasal cushion of claim 1 or 2, wherein the or each of the bellows respectively extend in length in an at least partially distal direction from each of the left and right lateral ends of the cushion.

4. The nasal cushion of any one of claims 1-3, wherein the or each of the bellows respectively extend in length both distally and medially from each of the left and right lateral ends of the cushion.

5. The nasal cushion of any one of claims 1-4, wherein the or each of the bellows respectively extend in depth into the cushion in a distal direction from each of the left and right lateral ends of the cushion.

6. The nasal cushion of any one of claims 1-5, wherein the connecting wall includes an upper portion connecting respective upper parts of the inner cushion wall and the outer cushion wall and a lower portion connecting respective lower parts of the inner cushion wall and the outer cushion wall, and the or each of the bellows extend in length along the upper portion of the connecting wall.

7. The nasal cushion of claim 6, wherein the or each of the bellows extend in length along both the upper portion and lower portion of the connecting wall.

8. The nasal cushion of claim 6 or 7, wherein the or each of the bellows extend in length along the connecting wall around respective left and right lateral ends of the cushion between the or a upper portion and the or a lower portion of the connecting wall.

9. The nasal cushion of any one of claims 6-8, wherein the or each of the bellows extend in length along the upper portion of the connecting wall and the respective left and right ends of the cushion such that a proximal end of the or each of the bellows is located closer to the lower portion of the connecting wall than to the upper portion of the connecting wall.

10. The nasal cushion of any one of claims 6-9, wherein each of the left and right lateral ends of the cushion have a proximal tip relative to a patient in use, and the or each of the bellows extend in length along the upper portion of the connecting wall above each of the respective proximal tips and terminate below each of the respective proximal tips.

11. The nasal cushion of any one of claims 6-10, wherein the or each of the bellows extend in length along the lower portion of the connecting wall more distally than an alar facial groove of a patient in use.

12. The nasal cushion of any one of claims 1-11, wherein or each of the each of the bellows comprise an inner bellows wall, an outer bellows wall, and a bellows walls joint connecting the inner and outer bellows walls.

13. The nasal cushion of claim 12, wherein the or each inner bellows wall is adjacent to the inner cushion wall and the or each outer bellows wall is adjacent to the outer cushion wall.

14. The nasal cushion of claim 12 or 13, wherein the or each of the inner bellows walls and outer bellows walls extend at one end from the connecting wall and at the other end from a respective bellows walls joint.

15. The nasal cushion of claim 12 or 13, wherein the or each of the inner bellows walls and outer bellows walls extend at one end from the upper portion of the connecting wall and at the other end from a respective bellows walls joint.

16. The nasal cushion of any one of claims 12-15, wherein in an at rest condition, the or each of the internal and external bellows walls extend into the cushion in a space between the internal cushion wall and external cushion wall.

17. The nasal cushion of any one of claims 12-16, wherein the or each bellows walls joint comprises a curved joint wall extending between respective inner and outer bellows walls.

18. The nasal cushion of any one of claims 12-17, wherein the or each bellows walls joint extends tangentially from each of an associated inner and outer bellows walls and is continuously curved therebetween.

19. The nasal cushion of any one of claims 12-18, wherein in an at rest condition there is a substantially uniform spacing between the inner and outer bellows walls of the or each of the bellows.

20. The nasal cushion of any one of claims 12-19, wherein in an at rest condition, the inner and outer bellows walls of the or each of the bellows are substantially plane-parallel with each other.

21. The nasal cushion of any one of claims 12-20, wherein a spacing between the inner and outer bellows walls of the or each of the bellows is about 0.5 mm to about 2 mm.

22. The nasal cushion of any one of claims 12-20, wherein in an at rest condition, the inner and outer bellows walls of the or each of the bellows are at least partially in contact with each other.

23. The nasal cushion of any one of claims 12-22, wherein the or each bellows walls joint has a relatively greater thickness than each of the associated inner and outer bellows walls.

24. The nasal cushion of any one of claims 12-23, wherein the or each bellows walls joint has a relatively greater stiffness than each of the associated inner and outer bellows walls.

25. The nasal cushion of any one of claims 12-24, wherein an upper bellows joint is defined between the or each of the inner and outer bellows walls and the connecting wall, and each upper bellows joint has a relatively greater thickness than each of the associated inner and outer bellows walls.

26. The nasal cushion of claim 25, wherein each of the upper bellows joint of the or each of the bellows has a relatively greater stiffness than each of the inner and outer bellows walls of the respective bellows.

27. The nasal cushion of any one of claims 12-26, wherein each of the inner and outer bellows walls are relatively thicker than at least an adjacent part of the inner cushion wall.

28. The nasal cushion of any one of claims 12-27, wherein a thickness of each of the inner and outer bellows walls is substantially uniform.

29. The nasal cushion of any one of claims 12-28, wherein a or the thickness of each the inner and outer bellows walls of the or each of the bellows are substantially equal.

30. The nasal cushion of any one of claims 12-29, wherein a or the thickness of each of the inner and outer bellows walls is about 0.3 mm to about 1 mm.

31. The nasal cushion of any one of claims 12-29, wherein a or the thickness of each of the inner and outer bellows walls is about 0.5 mm.

32. The nasal cushion of any one of claims 12-31, wherein the or each of the bellows comprise a first region in a central portion of the cushion and a second region extending to a proximal opening of the bellows, wherein an orientation of the bellows along their length changes from the first region of the bellows to the second region of the bellows.

33. The nasal cushion of claim 32, wherein in the first region the or each of the bellows extend in length proximally and curve medially along the connecting wall from the central portion of the cushion.

34. The nasal cushion of claim 32 or 33, wherein in the second region of the bellows, towards the proximal opening of the or each of the bellows, the bellows extend laterally.

35. The nasal cushion of any one of claims 32-34, wherein the proximal opening of the or each of the bellows is located towards a lateral side of each respective wing of the cushion.

36. The nasal cushion of any one of claims 32-35, wherein the proximal opening of the or each of the bellows is located closer to the outer cushion wall than to the inner cushion wall.

37. The nasal cushion of any one of claims 12-36, wherein the length of the or each of the bellows is between a first end and a second end of the or each of the bellows along the connecting wall, and the depth of the or each of the bellows is between an associated bellows walls joint and the connecting wall, and the depth of the or each of the bellows varies between its first end and its second end.

38. The nasal cushion of claim 37, wherein the depth of the or each of the bellows increases away from its first end and second end.

39. The nasal cushion of claim 37 or 38, wherein the depth of the or each of the bellows reduces to zero at one or both of its respective first end and second end.

40. The nasal cushion of any one of claims 37-39, wherein the depth of the or each of the bellows between its first and second ends is greatest at a region to be located between a supra-alar crease and an alar base of a patient's nose.

41. The nasal cushion of any one of claims 37-30, wherein the depth of the or each of the bellows between its first and second ends is greatest at a region to be located adjacent a or the alar groove of a patient's nose.

42. The nasal cushion of any one of claims 37-41, wherein the or each of the bellows extend in length around the left and right ends of the cushion, and a radius of curvature of the connecting wall at each of the left and right ends of the cushion is greater than a radius of curvature of each of the respective bellows walls joints at each of the left and right ends of the cushion.

43. The nasal cushion of any one of claims 1-42, wherein a maximum depth of the or each of the bellows is about 2 mm to about 20 mm.

44. The nasal cushion of any one of claims 1-42, wherein a maximum depth of the or each of the bellows is about 5 mm to about 10 mm.

45. The nasal cushion of any one of claims 1-42, wherein a maximum depth of the or each of the bellows is about 8.5 mm.

46. The nasal cushion of any one of claims 12-42, wherein the inner bellows wall or walls is/are substantially planar.

47. The nasal cushion of claim 46, wherein the inner bellows wall or walls is/are substantially planar but follow a shape of the bellows along the length of the or each of the bellows.

48. The nasal cushion of any one of claims 12-42, 46, and 47, wherein a spacing of the inner cushion wall from the or each of the adjacent inner bellows walls is variable over a depth of the or each of the bellows.

49. The nasal cushion of claim 48, wherein at a first region of the cushion the spacing of the inner cushion wall from an inner bellows wall is greater than the spacing of the inner cushion wall from the same inner bellows wall at a second region of the cushion.

50. The nasal cushion of claim 48 or 49, wherein at each of the left and right lateral ends of the cushion the spacing of the inner cushion wall from the inner bellows wall increases from each associated bellows walls joint to the connecting wall.

51. The nasal cushion of claim 50, wherein the spacing of the inner cushion wall from the inner bellows wall at each of the left and right wings of the cushion is locally increased towards the connecting wall at a part of the cushion to be located adjacent an alar groove of a patient.

52. The nasal cushion of any one of claims 49-51, wherein the inner cushion wall comprises two nasal bulges, each nasal bulge projecting medially of the cushion at a region to be located adjacent respective alar regions of a patient.

53. The nasal cushion of claim 52, wherein the two nasal bulges are located on respective left and right wings of the cushion and project towards each other.

54. The nasal cushion of claim 52 or 53, wherein the nasal bulges of the inner cushion wall are each located at a junction of the inner cushion wall with at least a proximal portion of the connecting wall of each wing of the cushion.

55. The nasal cushion of any one of claims 52 to 54, wherein the nasal bulges define a rim that extends along a or the junction of the inner cushion wall with at least a respective proximal portion of the connecting wall of each wing of the cushion.

56. The nasal cushion of any one of claims 1-55, wherein the inner cushion wall comprises a nasal aperture to receive a patient's nose.

57. The nasal cushion of claim 56, wherein the inner cushion wall comprises two lip bulges, each lip bulge projecting proximally of the cushion from respective portions of the inner cushion wall below the nasal aperture.

58. The nasal cushion of claim 57, wherein the lip bulges are spaced apart from each other across the central portion of the cushion.

59. The nasal cushion of claim 57 or 58, wherein the lip bulges are located on the inner cushion wall adjacent a junction of the inner cushion wall with a or the lower portion of the connecting wall.

60. The nasal cushion of any one of claims 1-59, wherein the or each of the bellows is able to independently expand to maintain a sealing engagement of the inner cushion wall with a patient's face across a range of lateral displacements of the patient's face relative to the outer cushion wall.

61. The nasal cushion of any one of claims 1-60, wherein the cushion is internally pressurised by a therapy airflow and the or each of the bellows expand when an external pressure resulting from a contact force of a patient's face at a bellows-adjacent portion of the inner cushion wall is less than the internal cushion air pressure.

62. The nasal cushion of any one of claims 1-61, wherein the or each of the bellows are biased towards a collapsed or at least partially collapsed position.

63. The nasal cushion of any one of claims 1-61, wherein at least one of the or each of the bellows is biased towards an expanded condition.

64. The nasal cushion of any one of claims 1-61, wherein the or each of the bellows is biased to a partially expanded position, such that upon a lateral displacement of a patient's face relative to the outer cushion wall the bellows at one wing of the cushion is caused to expand and the bellows at the other wing of the cushion is caused to contract.

65. The nasal cushion of claim 64, wherein the contraction of the or each of the bellows reduces a deformation or wrinkling of the inner cushion wall upon a lateral displacement of a patient's face relative to the outer cushion wall.

66. A respiratory interface comprising the cushion of any one of claims 1-65 and two side arms to support the non-face contacting outer cushion wall of the cushion, wherein in use an expansion of the or each of the bellows comprises a movement of a respective portion of the inner cushion wall from an adjacent portion of the outer cushion wall.

67. The respiratory interface of claim 66, wherein an expansion of the or each of the bellows cause an associated portion of the inner cushion wall to move medially.

68. The respiratory interface of claim 66 or 67, wherein an expansion of the or each of the bellows cause an associated portion of the inner cushion wall to move medially and proximally.

69. The nasal cushion of any one of claims 1-68, wherein the inner cushion wall includes an aperture for passing respiratory gases to a patient's nares.

70. The nasal cushion of claim 69, wherein a thickness of the inner cushion wall at a lip contacting part is increased relative to a remainder of the inner cushion wall.

71. The nasal cushion of claim 69 or 70, wherein a thickness of the inner cushion wall above the aperture is substantially uniform, and below the aperture a thickness of the inner cushion wall is relatively increased.

72. The nasal cushion of any one of claim 69-71, wherein a thickness of the inner cushion wall above the aperture is about 0.3 mm.

73. The nasal cushion of any one of claims 69-71, wherein a thickness of the outer cushion wall is about 0.5 mm.

74. The nasal cushion of any one of claims 1-73, wherein the bellows extending along the connecting wall at each of the left and right wings of the cushion is a single unitary bellows.

75. The nasal cushion of any one of claims 1-74, wherein the bellows extending along the connecting wall at each of the left and right wings of the cushion comprise a first bellows of the left wing and a second bellows of a right wing of the cushion.

76. The nasal cushion of claim 75, wherein the first bellows and second bellows are each provided along the upper portion of the connecting wall but are spaced apart from each other at the central portion.

77. The nasal cushion of claim 75 or 76, wherein the first bellows and second bellows are spaced apart across the central portion by about 1 mm to about 30 mm.

78. The nasal cushion of any one of claims 75-77, wherein the first bellows and second bellows are spaced apart across the central portion by about 1 mm to about 16 mm.

79. A nasal cushion for a respiratory interface, the cushion comprising an inner cushion wall to contact the a patient's face, a non-face contacting outer cushion wall and a first and second bellows located between the inner cushion wall and outer cushion wall,
wherein the bellows are each expandable to maintain a sealing engagement of the inner cushion wall with the patient's face under lateral movement of the patient's face relative to the outer cushion wall.

80. The nasal cushion of claim 79, wherein the bellows are laterally spaced apart on the cushion.

81. The nasal cushion of claim 79 or 80, wherein the cushion defines a central portion and two lateral wings, and the bellows are located between the inner cushion wall and outer cushion wall along at least an upper portion of each of the two lateral wings of the cushion.

82. The nasal cushion of claim 81, wherein the bellows extend in depth into the cushion from a most proximal part of each of the two lateral wings.

83. The nasal cushion of claim 81 or 82, wherein the bellows extend in depth into the cushion from a most peripheral part of each of the two lateral wings.

84. A nasal cushion for a respiratory interface, the cushion comprising an inner cushion wall to contact a patient's face, a non-face contacting outer cushion wall and a first and second bellows located between the inner cushion wall and outer cushion wall,
wherein the first bellows is expandable to maintain a sealing engagement with the patient's face when the patient lies on a side of their head associated with the second bellows.

85. The nasal cushion of claim 84, wherein the bellows are laterally spaced apart on the cushion.

86. The nasal cushion of claim 84 or 85, wherein the cushion defines a central portion and two lateral wings, and the bellows are located between the inner cushion wall and outer cushion wall along at least an upper portion of each of the two lateral wings of the cushion.

87. The nasal cushion of any one of claims 84 to 86, wherein the second bellows is expandable to maintain a sealing engagement with a patient's face when the patient lies on a side of their head associated with the first bellows.

88. A nasal cushion for a respiratory interface, the cushion having a central portion and left and right wings that extend to respective left and right ends from the central portion, the cushion comprising:
an inner cushion wall to contact a patient's face,
a non-face contacting outer cushion wall, and
a connecting wall that connects the inner cushion wall and outer cushion wall,
wherein the inner cushion wall comprises at least one bulge, each at least one bulge located at a junction of the inner cushion wall with the connecting wall.

89. The nasal cushion of claim 88, wherein each at least one bulge comprises a projection of the inner cushion wall away from an interior of the cushion.

90. The nasal cushion of claim 88 or 89, wherein the at least one bulge includes two nasal bulges, one nasal bulge on the junction of the inner cushion wall with the connecting wall towards the each of the left and right ends of the cushion.

91. The nasal cushion of claim 90, wherein the nasal bulges extend along the junction of the inner cushion wall with the connecting wall adjacent both an upper portion of the connecting wall and a proximal portion of the connecting wall.

92. The nasal cushion of claim 90 or 91, wherein the nasal bulges each define a medially projecting rim of the inner cushion wall along the junction of the inner cushion wall with the connecting wall about at least part of the ends of each of the left and right wings.

93. The nasal cushion of any one of claims 88-92, wherein the at least one bulge includes two lip bulges, the lip bulges each located either side of the central portion of the cushion at the junction of the inner cushion wall with a lower portion of the connecting wall.

94. The nasal cushion of claim 93, wherein the inner cushion wall comprises a nasal aperture to receive a patient's nose, and the two lip bulges each extend proximally along the junction of the inner cushion wall with the lower portion of the connecting wall from respective locations beneath the nasal aperture.

95. The nasal cushion of claim 94, wherein the two lip bulges each extend proximally from below a respective left and right sides of the nasal aperture.

96. The nasal cushion of any one of claims 88-95, wherein the cushion further comprises a bellows extending in length along the connecting wall and in depth into the cushion at each of the left and right wings of the cushion.

97. The nasal cushion of claim 96, wherein the or each of the bellows are expandable to increase a spacing between the inner cushion wall and outer cushion wall.

98. A nasal cushion for a respiratory interface, the cushion having a central portion and left and right wings that extend to respective left and right lateral ends from the central portion, the cushion comprising:
an inner cushion wall to contact a patient's face,
a non-face contacting outer cushion wall,
a connecting wall that connects the inner cushion wall and outer cushion wall, and
a bellows, the bellows extending in length along the connecting wall and in depth into the cushion at each of the left and right wings of the cushion,
wherein the bellows is expandable to increase a spacing between the inner cushion wall and outer cushion wall.
